(19) United States
(12) Patent Application Publication
(10) Pub. No.: US 2020/0041489 A1
(43) Pub. Date: Feb. 6, 2020

(54) HIGH PRECISION MICROPURIFICATION SYSTEM AND METHODS OF USE

(71) Applicants: Michael R. EMMERT-BUCK, Clarkston, MI (US); Michael Anthony TANGREA, Baltimore, MD (US)

(72) Inventors: Michael R. EMMERT-BUCK, Clarkston, MI (US); Michael Anthony TANGREA, Baltimore, MD (US)

(21) Appl. No.: 16/603,512

(22) PCT Filed: Apr. 6, 2018
(86) PCT No.: PCT/US2018/026469

§ 371 Date: Oct. 7, 2019

Related U.S. Application Data
(60) Provisional application No. 62/642,872, filed on Mar. 14, 2018.
(60) Provisional application No. 62/482,833, filed on Apr. 7, 2017.

(51) Int. Cl. G01N 33/50; C12Q 1/6804; C12Q 1/6806; C12Q 1/6841

(52) U.S. Cl. CPC G01N 33/5005; C12Q 1/6841; C12Q 1/6806; C12Q 1/6804

(57) ABSTRACT

Methods, techniques, and kits are provided herein for purifying cells using molecular targeting, at a cellular or subcellular level. The techniques comprise labeling a biological sample comprising cells with a probe capable of producing a protective barrier. The barrier is deposited onto the surface of the labeled cells or structures, to protect and retain the biological material under the barrier. A micropurification solution is applied to the biological sample, wherein the micropurification solution degrades, digests, or otherwise processes cells not covered by the barrier, allowing isolation of the target cells. In some aspects, a plurality of probes, each specific to a different target, may be used. The techniques may be performed without the need for complex instrumentation involving microscopy.

### CROSS-REFERENCE TO RELATED APPLICATIONS

This application claims priority to U.S. Provisional Application Ser. No. 62/482,833, filed Apr. 7, 2017, and to 62/642,872, filed Mar. 14, 2018, the contents of which are hereby incorporated by reference in their entireties.

### FIELD OF THE INVENTION

Histology methods and systems are provided that target and allow isolation of specific types of cells or structures using novel high-precision processing techniques.

### BACKGROUND

Biological samples such as histological tissue sections are inherently complex, containing numerous cell types (e.g., nerve cells, vasculature cells, epithelium cells, fibroblasts, etc.) as well as extracellular structures (e.g., extracellular matrix, etc.) that enable their specialized physiological functions. Quantitative molecular analysis of cells from histological slides is useful in developing a deeper understanding of fundamental pathophysiology and in generating effective clinical interventions [1-15]. While this complexity enables specialized functions of each tissue type, it can confound scientific or clinical analysis of individual types of cells within the tissue microenvironment.

For pathological diseases such as cancer, isolating tumor cells is further complicated by tumor heterogeneity, as a histology sample may comprise normal cells and cancerous cells, as well as regions of hyperplasia or neoplasia. Additionally, a tumor focus may contain multiple clones arising during tumor development and progression, with each clone having a unique expression or mutation profile. Thus, a histological tissue section may include a heterogeneous population of normal cells, a heterogeneous population of tumor cells and other structures present within the tissue and/or tumor microenvironment that complicate analysis.

In the early 1990s, Shibata demonstrated that cells coated with ink by a mechanical process were protected from UV radiation (see, Shibata et al., Am. Journ. of Path. (1992) vol. 141, no. 3, pp. 539-543). However, this technique was not cell-specific, typically covering groups of cells and protecting any cell covered by ink regardless of its particular cell type.

Several other strategies were developed in the 1990s and early 2000s to isolate cells from heterogeneous tissue sections, including manual scraping, micromanipulators, laser capture microdissection (LCM), and mesodissection [16-32]. Manual scraping involves removal of cells (e.g., using a razor, scraper, or other suitable tool) in a specific region of the tissue slide, and is typically performed under a microscope. While this technique is low cost, spatial resolution is low and non-target cell types may be captured along with the target cells. Micromanipulators have also been used to manually remove cells, as part of a process that also is typically performed under a microscope.

Microdissection techniques have also been used to isolate regions of cells from a tissue or histology slide. Such techniques may utilize immunohistochemistry (IHC)-based methods to identify target cells in order to achieve improved yield and precision. For example, immuno-laser capture microdissection has been used as a guide to the dissection process (see, Emmert-Buck et al., Science (1996), vol. 274: 8, pp. 998-1001). Other immunodissection methods also have been developed, including computer-based stain recognition software programs, expression microlabeling (xML), expression microdissection (xMD) (see, Hanson et al., Nature Protocols (2011) vol. 6: 4, pp. 457-467), AutoScanXT Software, etc. While such laser microdissection methods maintain spatial resolution suitable for isolating cells in heterogeneous environments, such techniques remain costly, are labor and time intensive, are performed manually typically under a microscope, and are subject to operator error. Mesodissection platforms offer yet another platform to isolate cells, by utilizing microfluidics tools to extract cells from particular regions of formalin-fixed paraffin-embedded tissue. However, mesodissection also is typically performed manually and prone to operator error, and is subject to contamination.

While laser-based dissection technology has greatly advanced modern biology and molecular pathology [8, 18, 33-46], in spite of these successes, laser dissection instruments do not offer an easy way to isolate target cells, and fail to meet the needs of modern molecular pathology and personalized medicine. Moreover, laser dissection devices are expensive to purchase and maintain, are time and labor-intensive to use, and are incapable of efficiently dissecting samples at a cellular level of precision.

With the advent of personalized medicine and in view of the heterogeneity of different types of tumors, techniques to accurately isolate specific types of tumor cells from individual patients for further analysis and with minimal human intervention are needed. Additionally, inexpensive options are needed for investigators or clinicians who lack access to commercial dissection instruments, and such options should allow procurement of cells by cell type in a precise manner.

Accordingly, while the aforementioned techniques are used, or have been used, for microdissection, they are subject to a variety of drawbacks, are not precise, and are generally not optimized for high throughput techniques involving personalized medicine. As the fields of biological research, molecular pathology, and precision medicine continue to evolve and coalesce, novel techniques will be needed to isolate and study the molecular content of specific cell populations in order to better understand biology and disease processes.

### SUMMARY

Methods, techniques, kits, and systems for isolating and collecting cells at a cellular/subcellular level of precision are provided herein. A biological sample comprising cells is labeled with a specific probe that directly carries or produces a protective barrier that is deposited onto the cells to which the probe is bound, forming a physical and/or molecular barrier that selectively covers the labeled cells and does not cover unlabeled cells. A micropurification solution is applied to the biological sample that selectively degrades or differentially processes (e.g., contains an RNase that degrades RNA) cells or cellular components not covered by the protective barrier. The labeled cells covered with the barrier are not degraded or processed by the micropurification solution, or are degraded or processed differentially, e.g., in a different manner or to a different degree that allows recovery of the labeled cells and not the unlabeled cells.

After the micropurification solution is added, both the nontarget cells/structures and the target cells/structures may be separately recovered. The nontarget cells are solubilized or otherwise differentially processed by the micropurification solution and may be recovered in the solution. The target cells remain on the slide due to the microprotection effect of the barrier and can be subsequently recovered by slide scraping, further chemical processing steps, or other recovery means.

Specific cells or structures can be recovered from the biological sample (e.g., from the histological slide) because the probe binds to specific target cell(s) or structures based upon their physical or molecular characteristics, e.g., cell surface antigens, nucleotide sequences, etc.). These characteristics allow target cells to be specifically labeled by a probe (e.g., an antibody, a nucleotide-based hybridization probe, an affimer, an aptamer, etc.) that produces a precipitate or other physical or chemical barrier that protects the labeled cells from a micropurification solution that degrades or otherwise processes uncovered cells. The high precision of the labeling leads to a corresponding high precision in the location of the protective barrier, and this precision allows isolation of a desired cell population with high resolution compared to prior methods. Moreover, because the method is probe-based it is not dependent upon a human operator to manually select target cells of interest.

It is to be understood that this Summary is not intended to identify key or essential features of embodiments of the present disclosure, nor is it intended to be used to limit the scope of the present disclosure. Other features of the present disclosure will be apparent from the description below.

### BRIEF DESCRIPTION OF THE DRAWINGS

FIGS. 1A-1E show images of various prostate tissue (FIG. 1A) with differing morphologies: normal (FIG. 1B), hyperplasia (FIG. 1C), prostatic intraepithelial neoplasia (FIG. 1D), and carcinoma (FIG. 1E). As shown by this series of images, a biological sample such as prostate tissue may include cells in a variety of different states with different characteristics. Accordingly, isolating a single type of cell is often difficult.

FIG. 2 shows an illustration of a biological sample, e.g., obtained by biopsy. The biological sample may contain fibroblasts (a type of normal cell), tumor cells, inflammatory cells (e.g., cells from the immune system including B and T cells), as well as vasculature and other structural components. Accordingly, not only may a type of cell, such as prostate cells, be present in various states, but also, other cell types (e.g., normal, tumor, immune, etc.) may be present in a sample.

FIGS. 3A-3E show schematic drawings of various stages of the Histology Micro Protection (HMP) method provided herein. The uppermost panel (FIG. 3A) shows a non-immunostained, non-labeled histological section on a glass slide. The cells and structure are represented by an oval filled with crosshatched lines. Unique physical or molecular characteristics of the target cell(s)/structures allow specific labeling by a probe, as shown in FIG. 3B, wherein target cells labeled with the probe are shown as solid black ovals or circles. The probe produces a chemical material that protects the cells (the microprotection effect) from subsequent processing as shown in FIG. 3C. The labeled cells may be incubated in a micropurification solution (shown as diagonal lines), which removes or differentially processes unlabeled cells and structures from the slide, for about 10 minutes, although other amounts of time may be suitable as well (e.g., up to 60 minutes or any amount of time in between, etc.). FIG. 3D is an illustration of the biological sample after processing. At this stage, the solubilized non-target cells are recovered by collection of the micropurification solution (shown as diagonal lines), e.g., into a test tube. As shown by FIG. 3E, the labeled target cells, which remain on the slide due to the microprotection effect, may be recovered by collecting (e.g., scraping) the cells (shown as crosshatched lines) into a test tube. Thus, FIG. 3D demonstrates negative selection with regard to the target cells, in which cells that are unlabeled are solubilized and removed from the slide, allowing a subsequent collection of the labeled cells.

FIGS. 4A-4D show another illustration of the HMP method. FIG. 4A shows a tissue section mounted on a glass slide. In this example, the probe may be antibody-based. A first primary antibody, having specificity for a specific antigen associated with a particular type of cell or subcellular component, is applied under conditions suitable for antibody binding to the antigen. A secondary antibody is then applied (also shown in FIG. 4A), which binds to the first primary antibody. The secondary antibody may be conjugated or may form a complex with an enzyme capable of reacting with a substrate to form an insoluble chemical material (as shown in FIG. 4B using crosshatched lines). The insoluble chemical material, or precipitate, covers the cells to which the probe is attached. FIG. 4C shows application of the micropurification solution (shown as diagonal lines) to the histology slide, and the unprotected cells (the cells not bound to a probe) are solubilized. Upon removal of the processing buffer, the labeled, covered cells remain on the slide, as shown in FIG. 4D, and may be collected by scraping or other suitable means.

FIGS. 5A-5F show a series of microscopic images for histological sections of multiple cases of colorectal cancer before and after HMP. At FIG. 5A, colon carcinoma tissue is immunostained using cytokeratin AE1/AE3 antibodies (2× magnification). The cytokeratin AE1/AE3 antibodies bind to various cytokeratins in formalin-fixed, paraffin embedded tissue. For example, AE1 detects high molecular weight cytokeratins 10, 14, 15, and 16, and low molecular weight cytokeratin 19, while AE3 detects high molecular weight cytokeratins 1, 2, 3, 4, 5, and 6, and low molecular weight cytokeratins land 8. DAB is provided as a substrate, which forms an insoluble brown precipitate upon oxidation, e.g., by a peroxidase enzyme attached to or part of the probe. The target cancer cells are darkly stained while the non-target cells/structures (e.g., stroma, fibroblasts, inflammatory cells, nerves, etc.) are not darkly stained and thus are faintly visible between the stained targets. FIG. 5B shows the colon carcinoma tissue (from FIG. 5A) immunostained using cytokeratin AE1/AE3 antibodies after HMP. The stained target cells remain intact on the slide after HMP but the non-target cells were solubilized and were no longer present on the slide (2× magnification). FIG. 5C shows colon carcinoma tissue (same as FIG. 5B but at a 10× magnification) immunostained using cytokeratin AE1/AE3 antibodies after HMP.

FIG. 5D shows colon carcinoma tissue immunostained with antibodies to Ki-67 (at a 10× magnification). For example, the MIB1 IgG1 antibody, which binds to Ki67 in formalin-fixed, paraffin embedded tissue, may be used. The target cancer nuclei are darkly stained while the non-target cells/structures (e.g., stroma, fibroblasts, inflammatory cells, nerves, etc.) are not stained and thus are faintly visible between the stained targets. FIG. 5E shows colon carcinoma tissue immunostained with antibodies to Ki-67 after HMP. The stained target nuclei remain intact on the slide after HMP but the non-targets were solubilized and were no longer present on the slide (10× magnification). FIG. 5F shows colon carcinoma tissue (same as FIG. 5E but with 20× magnification) immunostained with antibodies to Ki-67 after HMP.

FIG. 6 shows an agarose gel image of DNA amplification of a 129 base pair (bp) product from HMP target cells. The data indicate that DNA is present in the recovered target cells and can be successfully amplified by PCR after the HMP process.

FIGS. 7A-7B show microscopic images of human colon cancer before HMP (FIG. 7A) and after HMP (FIG. 7B). In this example, cytokeratin was selected as an antigen, and DAB was used to form an insoluble chemical material (brown) when oxidized, e.g., by a peroxidase enzyme conjugated to or complexed with the probe.

FIGS. 8A-8B show microscopic images of human colon cancer before HMP (FIG. 8A) and after HMP (FIG. 8B). In this example, cytokeratin was selected as an antigen, and DAB was used to form an insoluble chemical material (brown) when oxidized.

FIGS. 9A-9B show microscopic images of human colon cancer before HMP (FIG. 9) and after HMP (FIG. 9B). In this example, a cluster of differentiation antigen (pan-CD) was selected as an antigen to label lymphocytes, and DAB was used to form an insoluble chemical material (brown).

FIGS. 10A-10B show microscopic images of human colon cancer before HMP (FIG. 10A) and after HMP (FIG. 10B). In this example, cytokeratin was selected as an antigen, and DAB was used to form an insoluble chemical material (brown).

FIGS. 11A-11B show microscopic images of human colon cancer before HMP (FIG. 10A) and after HMP (FIG. 10B). In this example, pan-CD was selected as an antigen, and DAB was used to form an insoluble chemical material (brown).

FIGS. 12A-12B show microscopic images of human breast cancer before HMP (FIG. 12A) and after HMP (FIG. 12B). In this example, pan-CD was selected as an antigen, and DAB was used to form an insoluble chemical material (brown).

FIGS. 13A-13B show microscopic images of human colon cancer before HMP (FIG. 13A) and after HMP (FIG. 13B). In this example, epithelium membrane antigen (EMA) was selected as an antigen, and Fast Red was used to form an insoluble chemical material.

FIGS. 14A-14B shows microscopic images of human colon cancer before HMP (FIG. 14A) and after HMP (FIG. 14B). In this example, Ki-67 was selected as an antigen, and Fast Red was used to form an insoluble chemical material.

FIG. 15 shows a microscopic image of human colon cancer after HMP. In this example, Ki-67 was selected as an antigen, and DAB was used to form an insoluble chemical material.

FIG. 16 shows a list of samples which have been processed according to the HMP techniques provided herein. As shown in this figure, HMP was successfully used to isolate the tumor strains (see arrows), while the non-tumor cells were not detected.

FIG. 17 shows results of an IHC procedure with cases: PC2 (×2) and CRC22 (×2) followed by micropurification, RNA extraction, and microRNA Qubit reading. The primary antibody was 1:100 cytokeratin. Conditions for the micropurification buffer were 1 mg/ml proteinase K in Tris buffer with 1% SDS. The slide was scraped after IHC for RNA extraction, scraped after micropurification for RNA extraction, and the digest buffer from micropurification was collected for RNA extraction. Results, which are an average of triplicate reading, showed successful recovery of mRNA. A standard antigen retrieval procedure was used, e.g., microwave treatment using Citra buffer as described herein.

FIGS. 18A-18D show nuclei from two sets of experiments at different magnifications from human breast cancer FFPE tissue that was isolated using micropurification, and stained with a histone primary antibody and a secondary antibody labeled with alkaline phosphatase. The chromogen was NBT/BCIP.

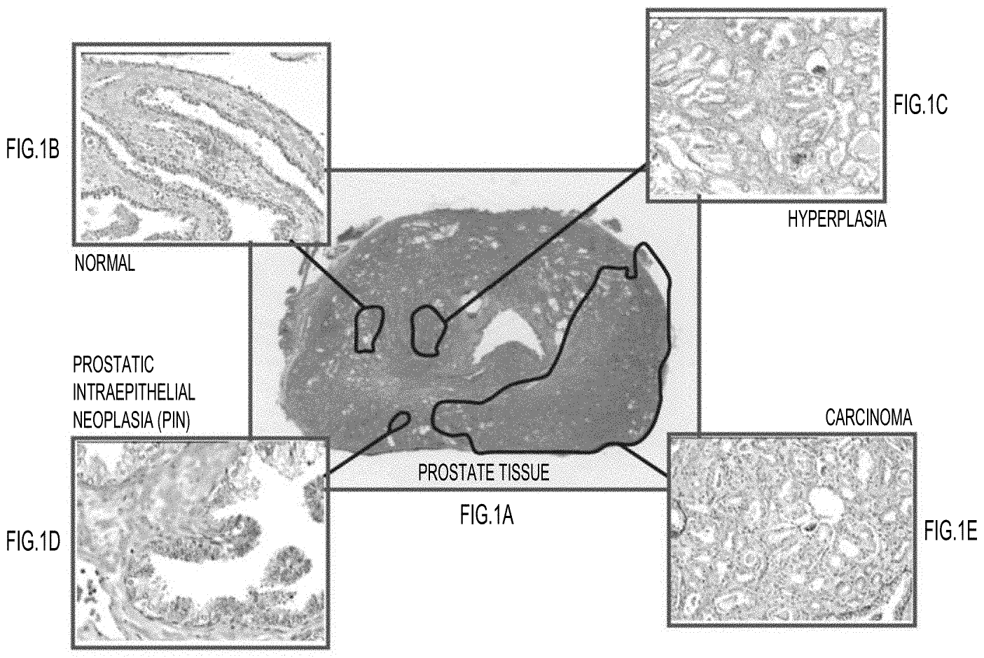

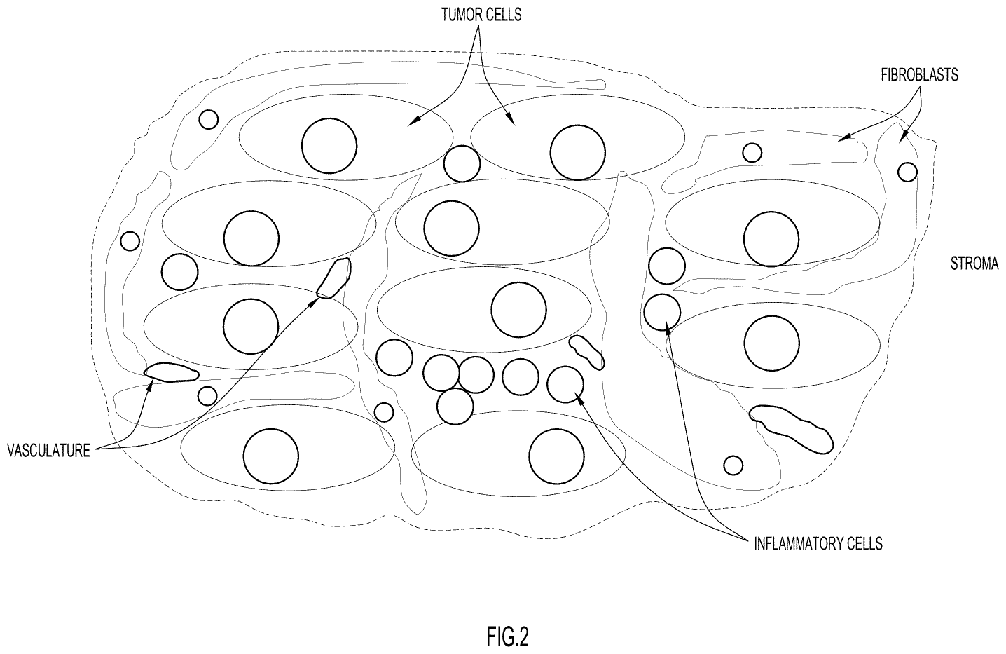

### DETAILED DESCRIPTION

Histology Micro Protection (HMP) methods and systems are provided that allow high resolution selection of cells (e.g., tumor cells) or subcellular structures at a molecular level using a probe that produces an chemical material that covers the cell to which the probe is attached. HMP utilizes and integrates novel probe-based targeting and microprotection effects to isolate specific types of cells or structures in a complex biological sample comprising a plurality of different cell types. The isolated cells can be subsequently analyzed by any of a variety of techniques, including, e.g., expression profiling, NGS, etc.

HMP methods provide increased resolution, higher throughput, and greater ease of use compared to current cell selection methods and technologies such as manual dissection, laser capture microdissection, or an immuno-based dissection method. For example, HMP can recover specific cells or subcellular structures from histology slides within minutes without relying on complex instrumentation involving laser dissection, slide irradiation, and/or microscopy. Using HMP, cells or subcellular structures are recovered based on their molecular characteristics. Accordingly, HMP reagents may be provided as part of a kit to provide cell selection capability in the absence of complex technologies. Additionally, HMP provides a high level of precision, allowing isolation of a specific type of cell with high yield, as compared to current techniques which typically rely on complex instrumentation and produce lower yields than HMP. These techniques may also be used to isolate a specific type of cell present at a low concentration within a biological sample.

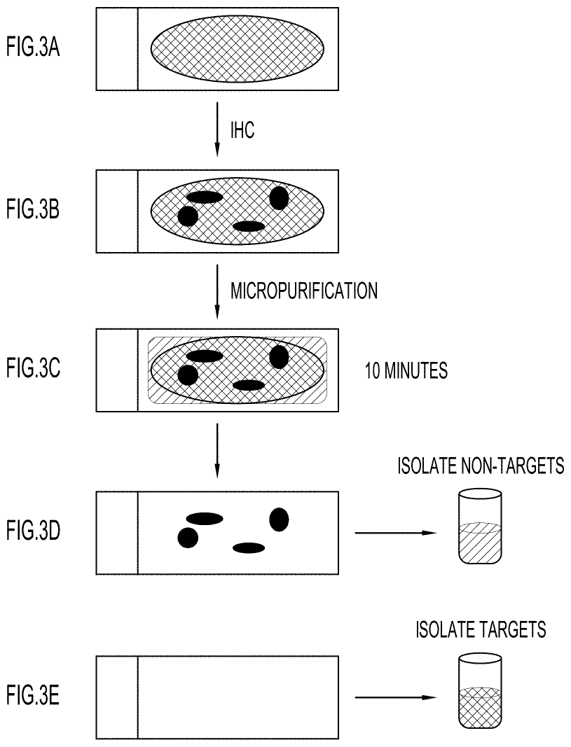

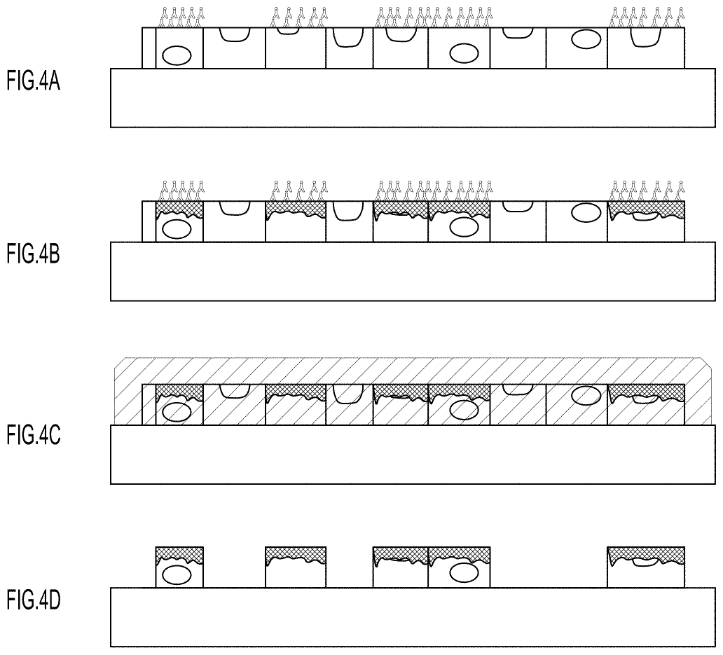

In one aspect, HMP may be used to isolate cells on a histological slide carrying a biological sample. The histological slide may be generated according to techniques known in the art. For example, a biological sample may be sectioned, formalin-fixed, and embedded in paraffin, and may undergo additional processing to expose the antigens (e.g., antigen retrieval) so that probes can bind to the antigen. The slide may then be incubated with the probe, wherein the probe attaches to cells of interest based upon the cell's molecular characteristics. A moiety on the probe forms, e.g., directly or as a byproduct, a barrier that coats the cells to which the probe is attached. The labeled cells may then be incubated in a micropurification solution for a period of time sufficient to solubilize, differentially process, and/or degrade cells that are not coated with the chemical material. The time required to solubilize, differentially process, and/or degrade cells may vary depending on the tissue but may be, for example, about 10 minutes. The attached labeled cells may then be removed (e.g., by scraping, etc.) into another container. This process is also shown in FIGS. 3A-3E and FIGS. 4A-4D. Additional aspects of this process are provided as follows.

### Histology, Cytology, and Cell Culture Slides, Flasks, and Petri Dishes

Histology samples typically are thinly sliced, or sectioned, such that the individual sections may be stacked (vertically) to reconstruct the 3-dimensional structure of the biological sample. Accordingly, in some aspects, the histological slide may include extracellular targets, targets on the surface of a cell, or targets within the cell (e.g., the cytoplasm, the nucleus, etc.) that are accessible by the probes.

In some aspects, the cells are not processed according to histological techniques, but are adhered to other surfaces, including but not limited to, plastic, glass, polymeric surfaces such as gels, etc. Cells may be in a variety of states (not limited to formalin-fixed and/or paraffin-embedded), including frozen sections, live cells, cultured cells, cells subjected to suspension culture, etc.

Paraffin-embedded tissues on slides may be dewaxed using xylenes, as referenced in the experimental protocols provided herein. Any suitable organic solvent may be used, including, but not limited to xylene(s) benzene, toluene, biphenyl, and the like. Further, the tissue slide may be placed in the organic solvent from 1 minute up to 30 minutes, or any range in between, or more. After dewaxing the sample can be rehydrated as necessary, using methods that are well-known in the art, including using solution containing decreasing concentrations of ethanol or another water-miscible solvent. Any suitable buffer may be utilized (e.g., Tris, etc.), and the protocol is not intended to be limited to Citra Plus buffer.

### Antigen Retrieval

Formalin-fixed tissues sometimes require an antigen retrieval step before a probe can specifically bind to cells in the tissues. Cross-linking during fixation can mask antigenic sites and antigen retrieval methods can reverse, at least partially, this cross-linking and expose antigenic sites, allowing probes to bind. Methods of antigen retrieval are well known in the art, and the specific protocols provided herein are not intended to be limiting of the methods that can be used. For example, the slides may be microwaved at maximal power (Sanyo, 1200 Watts) until boiling, wherein boiling may occur for any suitable amount of time, e.g., from 15 seconds up to 10 minutes or more, or any range in between. The slides may then be microwaved for an additional amount of time at medium power, wherein the amount of time may range from 1 minute up to 20 minutes, or more, or any range in between. The slides may be cooled for a period of time ranging from 5 minutes up to 30 minutes, or more, or any range in between, followed by rinsing with diH₂O.

A single heating step, or multiple heating steps may be used. Any combination or duration of heating steps, e.g., time, temperature, cycles, may be used provided that antigens are exposed and can be bound by a probe, such as a primary antibody, and that damage to the underlying biological material of the target cells is minimized

### Antigens

A variety of antigens may be used with the HMP methods as described herein. In general, the HMP method may be applied to any biological target, e.g., including but not limited to an extracellular target, a target on the surface of the cell, a intracellular target, a lipid, a protein, a cell type, a subcellular compartment, or any target that may be specifically labeled. A suitable antigen may be specific to a particular type of cell and/or may indicate a cancerous state of the cell (e.g., indicate high proliferation). For example, antigens associated with the presence of cancer include, but are not limited to: alpha fetoprotein (AFP), CA15-3, CA27-29, CA19-9, CA-125, calcitonin, calretinin, carcinoembryonic antigen (CEA), CD34, CD99MIC 2, CD117, chromogranin, chromosomes 3, 7, 17, and 9p21, cytokeratin (e.g., TPA, TPS, Cyfra21-1), desmin, epithelial membrane antigen (EMA), factor VIII, CD31 FL1; glial fibrillary acidic protein (GFAP), gross cystic disease fluid protein (GCDFP-15), HMB-45, human chorionic gonadotropin, immunoglobulin, inhibin, keratin, lymphocyte marker, MART-1 (Melan-A), myo D1, muscle-specific actin (MSA), neuron-specific enolase (NSE), placental alkaline phosphatase (PLAP), prostate-specific antigen (PSA), PTPRC (CD45), S100 protein, smooth muscle actin (SMA), synaptophysin, thymidine kinase, thyroglobulin (Tg), thyroid transcription factor-1 (TTF-1), tumor M2, and vimentin.

A single antigen may be used with the HMP methods provided herein, for example, an antigen unique to a specific type of immune cell. Alternatively, cocktails of antigens may be used, allowing isolation of multiple types of cells (e.g., the immune repertoire using CD 68, CD4, CD11, etc.). In some aspects, a cocktail of probes may be used to recover multiple types of lymphocytes, as part of a diagnostic workflow to ascertain immunological activation status in a patient's cancer. As another example, the cocktail may comprise a mix of antibodies that bind to CD6, CD8, CD20, and CD68. The methods and techniques provided herein are not intended to be limited to a particular antibody or combination of antibodies.

### Probes—Antibodies

In some embodiments, the probe may comprise an antibody that is specific for a particular antigen associated with a particular type of cell. In some aspects, the probe is conjugated or complexed with a reactive moiety, such as an enzyme or other inhibitor of a chemical process, or other reporter molecule capable of reacting with a substrate to produce an chemical material that is deposited on the cells to which the probe is bound. In general, the probe may comprise one or more antibodies, wherein at least one antibody is conjugated to an enzyme capable of reacting with a substrate to produce a protective chemical material. In some embodiments, under varying conditions, different amounts of chromogen may be deposited onto the surface of the cell. In some aspects, relatively light deposition may be sufficient for a protective effect. In other aspects, a relatively moderate deposition may be sufficient for a protective effect. In still other aspects, relatively heavy deposition may be sufficient for a protective effect. In yet other aspects, wherein a plurality of probes are used, each type of probe may generate a light deposition of chromogen that in combination with depositions from other probes are sufficient for a protective effect.

In some embodiments, the probe comprises an antibody to a specific antigen, with the antibody complexed or conjugated to an enzyme capable of processing a substrate to form a protective chemical material. In other embodiments, the probe comprises a primary antibody that binds to a tissue antigen and a second antibody that binds to the primary antibody, where the secondary antibody is covalently conjugated or non-covalently complexed to an enzyme capable of processing a substrate to form a protective chemical material.

As an alternative, enzyme inhibitors (e.g., inhibitors to the enzyme used for degradation, such as proteinase K, trypsin, etc.) or other inhibitors of chemical processes may be complexed or conjugated to the probe, e.g., via the secondary antibody. In this embodiment, the inhibitor may locally deactivate or inhibit the degradation or processing enzyme or other reagent so that target cells, covered by the probe are protected. In cases in which the degradation enzyme is capable of penetrating the chromogenic stain, a polymer, e.g., polyethylene glycol (PEG), etc., may be conjugated or complexed with the degradation enzyme or other chemical reagent so that the enzyme or reagent is sterically hindered from penetrating the chromogenic stain, and thus, the underlying stained cells remain protected.

Essentially any antibody may be selected as a primary antibody, provided that it specifically binds to an antigen associated with a target cell type. Methods of determining antibody specificity are well known in the art. An antibody typically is considered specific if it binds to a target antigen without binding to unrelated antigens. In some embodiments, the primary antibody may bind to an antigen associated with a type of normal cell. In other embodiments, the primary antibody may bind to an antigen associated with a type of cancerous or tumorigenic cell. In still other embodiments, the primary antibody may bind to an antigen associated with a type of immune cell.

In some aspects, the antibodies may bind to antigens present on the surface of the cell, to extracellular antigens, or antigens present within the interior of the cell (provided that the cell was cross-sectioned during slide preparation).

Examples of suitable antibodies include, but are not limited to: cytokeratin AE1/AE3 antibodies, anti-Ki-67 antibodies (Ki-67 is a nuclear antigen), anti-histone antibodies (histone is a nuclear antigen), anti-Epithelial Membrane Antigen (EMA) antibodies (EMA is a cell membrane antigen), anti-CD8 antibodies (CD8 is a lymphocyte antigen) anti-CD20 antibodies (CD20 is a lymphocyte antigen), anti-vimentin antibodies (vimentin is a stroma antigen), etc. In some aspects, cytokeratin AE1/AE3 antibodies may be a mixture of two different clones of anti-cytokeratin monoclonal antibodies, e.g., AE1 that detects high molecular weight keratins and AE3 that detects low molecular weight keratins. In other aspects, antibodies to mitochondrial antigens, antibodies to Golgi antigens, antibodies to nuclear antigens may be generated and used with the methods and systems provided herein.

Either a single probe or a cocktail of multiple probes can be utilized to protect cells, allowing flexibility and tunability with regard to experimental strategy. For example, in a histological section containing cancer cells, a probe can target the tumor cells during the micropurification process, thus separating the slide into two components; neoplastic cells versus all of the normal cell types (e.g., fibroblast, nerve, lymphocyte, etc.) and structural components (e.g., basal lamina, matrix, neo-vessels, etc.) present in the tumor microenvironment. If the probe(s) target tumor cells, then the normal cells and structural components are solubilized or otherwise processed, leaving behind the tumor cells. If a cocktail of probes target normal cells and structural components, then the tumor cells are solubilized or processed.

Suitable titers for antibodies include 1:10, 1:20, 1:50, 1:100, 1:200, 1:500, 1:1000, 1:2500, or any other amount.

In general, for antibody binding, any suitable incubation time may be used, provided that antigens exposed by antigen retrieval remain exposed and in a form in which antibody recognition and binding of the probe may occur. Examples of suitable incubation times for primary and/or secondary antibodies include, but are not limited to, about 15 minutes, about 30 minutes, about 45 minutes, about 60 minutes, or any range in between. In other aspects, incubation times with the secondary antibody may be about 15 minutes, about 30 minutes, about 45 minutes, about 60 minutes, or any range in between.

### Probes—Affimers

As another example, an affimer may be used as a probe. Affimers are small domain proteins that specifically bind to an antigen and typically exhibit high thermal and biophysical stability, and typically lack di-sulfide bonds or other post-translational modifications.

Affimers may be utilized with the HMP methods described herein. In some aspects, affimers may be biotinylated and detected using a streptavidin horseradish peroxidase (HRP) complex. For example, the horseradish peroxidase may oxidize DAB to produce a brown precipitate. In general, any suitable reporter molecule may be used.

### Probes—DNA

A further type of probe that may be used in the methods described herein is a chromogenic in situ hybridization (CISH) probe. CISH uses a complementary DNA or RNA strand to localize to a specific DNA or RNA sequence in a tissue specimen, and may be used to determine whether genetic mutations (e.g., gene deletion, chromosome translocation, gene amplification, etc.) are present. A combination of one or more antibody probes and one or more CISH probes allows identification and separation of cells and tissues that previously were difficult to visualize using the methods and systems described herein.

For example, a DNA probe may be generated that is complementary to a target DNA sequence of interest. The DNA probe may be labeled, for example with a digoxigenin label. A primary antibody can be used to bind to the probe (e.g., the primary antibody may be an anti-digoxigenin fluorescein isothiocyanate antibody). A secondary antibody may bind to the primary antibody, wherein the secondary antibody contains a reactive moiety capable of processing a reagent (e.g., the secondary antibody may be an anti-fluorescein-isothiocyanate horseradish peroxidase (HRP) antibody that reacts with DAB to produce and deposit a chromogenic signal (e.g., a dark brown precipitate) at the target site. Alternatively, the reactive moiety can be an alkaline phosphatase enzyme that is used to produce a chromogenic signal.

In general, the probe may comprise any molecule that binds to a target molecule, wherein the probe is capable of reacting with a reagent to produce a chemical product that is deposited on the probe labeled cells.

### Chemical Material

In embodiments of the methods described herein, a chemical material is deposited or placed on the surface of the target cells labeled with the probe to form a barrier. In some embodiments, the material may be attached to the probe or may be a product of the probe (e.g., from processing a substrate). The chemical material may be soluble or insoluble. For example, the material may be generated as a product of a chemical reaction, wherein a reagent is processed by an enzyme attached to the probe to generate an insoluble product. Suitable enzymes include horseradish peroxidase and alkaline phosphatase.

Reagents are that react with such enzymes are generally referred to as chromogenic reagents. Suitable reagents for reaction with horseradish peroxidase include, but are not limited to: 3-amino-9-ethylcarbazole (AEC); 3,3′-Diaminobenzidine (DAB) (with or without a nickel enhancer); and Tetramethyl benzidine (TMB); Suitable reagents for reaction with alkaline phosphatase include, but are not limited to 5-bromo-4-chloro-3indolyl-phosphate/nitrobluetetrazoleum (BCIP/NBT); Naphthol AS-MX phosphate+Fast Blue BB; Naphthol AS-MX phosphate+Fast Red TR; and Naphthol AS-MX phosphate+new fuchsin; and nitrobluetetrazoleum (NBT).

The secondary antibody typically is conjugated to an enzyme which reacts with the chromogenic reagent to produce a chemical material. For example, glucose oxidase reacts with NBT to produce a blue insoluble chemical material; alkaline phosphatase reacts with naphthol AS-MX phosphate+new fuschin to produce a red insoluble chemical material, with naphthol AS-MX phosphate+fast red to produce a red insoluble chemical material, with naphthol AS-MX phosphate+fast blue BB to produce a blue insoluble chemical material, and with BCIP/NBT to produce a blue insoluble chemical material. Horseradish peroxidase reacts with TMB to produce a blue insoluble chemical material, with DAB (with or without a nickel enhancer) to produce a brown or black insoluble chemical material; and with AEC to produce a red insoluble chemical material. Lists of chromogens, and the enzymes that react with the chromogens may be found at, e.g., http://www.abcam.com/kits/chromogens-and-enhancers.

The methods and systems provided herein are not limited to IHC stains, and therefore, any system with the capability of locally depositing a material in a cell-specific manner may be used with the micropurification techniques provided herein.

In other aspects, the present techniques may be used for ligand receptor binding studies. For example, the precision of the HMP process may be used to localize the protective effect to selected ligand and/or bound receptors in a specific subcellular region, recovering only the biomolecules (e.g. signaling molecules) in the immediate vicinity that are associated with the receptor.

### Tunability

The methods and systems provided herein are highly tunable, and may be configured to isolate a wide variety of targets, including types of normal cells, types of diseased cells (e.g., such as a cancerous or tumorigenic cell), as well as immune cells. Once isolated, the cells may be subjected to further analysis, including: NGS sequencing, expression profiling, proteomic analysis, lipid analysis, etc. In general, any target for which a suitable probe is provided, e.g., a probe which can specifically bind to the target and which is capable of reacting with a reagent to produce a protective barrier, e.g., a precipitate to cover the cell, may be isolated by the methods and systems provided herein.

In particular, the components of this process (e.g., probe, enzyme, chromogenic reagent, micropurification solution, buffers, etc.) may be individually tuned to a particular assay, and once the assay has been validated, the identified conditions (e.g., temperature, time, antigen retrieval processing, etc.) may be further optimized.

### Micropurification Solution

The micropurification solution may be used to degrade, digest or otherwise process cells that have not been protected by the protective barrier. The solution may contain at least one protease and, optionally, a detergent, and other enzymes and chemical reagents. For example, the micropurification solution, also referred to as a digestion buffer, may contain 1 mg/ml proteinase K and 2% SDS. When present, the SDS concentration may be from 0.01% SDS up to 5% SDS, or any amount in between.

The concentration of the protease used in the micropurification can be any concentration sufficient to degrade or process the cells not protected by the protective barrier. For proteinase K, the concentration may range from about 0.1 mg/ml to about 10 trig/mg, from about 0.5 mg/ml to about 5 mg/ml, or any amount in between. In some aspects the concentration of proteinase K is about 1 mg/mg, about 2 mg/ml, or about 3 mg/ml.

The slide is incubated in the digestion buffer for a time sufficient to digest the cells not protected with the barrier to the extent necessary to allow removal or processing of those cells. For example, the incubation time may be about 5 minutes, 10 minutes, 15 minutes, 30 minutes, 45 minutes, 60 minutes, 75 minutes, 90 minutes, 120 minutes or 150 minutes or any amount in between. For longer incubation times, additional buffer or diH₂O may be added so that the slides do not dry out. In some aspects, digestion/processing during micropurification may be complete in 10-20 minutes.

In general, any serine protease may be used, including but not limited to, proteinase K, trypsin, chymotrypsin, etc. In some embodiments, the micropurification solution comprises one serine protease, two serine proteases, or three or more serine proteases. In still other embodiments, degradation enzymes may be selected based upon the type of tissue to be analyzed, e.g., enzymes known to degrade muscle tissue and cells may be selected for slides containing muscle tissue, enzymes known to degrade adipose tissue and cells may be selected for slides containing adipose tissue, etc.

In general, any suitable detergent may be used, including but not limited to SDS, ADS, Triton X-100, Sarkosyl, Tween, etc. Further, in some aspects, two or more detergents may be used as part of the micropurification solution.

In general, any suitable buffer may be used, including but not limited to Citra Plus, Tris-HCl, citrate buffer, etc. Further, in some aspects, two or more buffers may be used as part of the micropurification solution.

The pH of the micropurification solution may range anywhere from a pH of 1 to a pH of 10. In general, the pH should match the optimal conditions of the micropurification enzyme.

In some embodiments, the barrier or shield may prevent, inhibit, or reduce degrading components present in the micropurification buffer (e.g., proteases, detergents, etc.) from physically contacting the targeted cells. In other embodiments, the barrier may act to inhibit or reduce activity of a degrading enzyme, e.g., such as a protease. In still other embodiments, the barrier may restrict or confine motion of the protected biological material to prevent or reduce exposure to the degrading enzymes.

In some aspects, the techniques and systems provided herein may be used to identify novel general or patient specific biomarkers. For example, analysis of the digested, processed, or solubilized portion of the slide may lead to the identification of novel serum biomarkers or validation of predicted biomarkers. Solubilized protein from microprotected cells can be analyzed via SDS-PAGE, western blot, reverse phase protein arrays, dot blots, mass spectrometry, etc.

### Kits

Kits may be provided to carry out the methods presented herein. The HMP kit may comprise any or all of the reagents necessary to carry out the methods described herein. The kit may comprise a micropurification solution, and optionally, a neutralization solution that deactivates enzymes present in the micropurification solution to halt micropurification. In other embodiments, the kit may comprise individual reagents which are combined to form the micropurification solution.

In some aspects, the kit may comprise a probe and a chromogenic reagent, which reacts with the probe to generate an insoluble chemical material, along with the micropurification solution. The probe may be a monoclonal antibody or other antigen binding molecule, which specifically binds to an antigen present in the biological sample. The probe may be conjugated to an enzyme capable of reacting with the reagent to produce the insoluble chemical material.

Kits also are provided comprising the micropurification solution, a second antibody or second antigen binding fragment thereof that specifically binds to a first monoclonal antibody or first antigen binding fragment thereof. In this example, the first monoclonal antibody or first antigen binding fragment may be custom generated (not part of the kit), and designed to bind to the second antibody or second antigen binding fragment. Custom mAbs may be produced, e.g., to bind to a specific markers associated with a cell, depending on the nature of the assay. The second antibody or second antigen binding fragment is conjugated to or complexed with an enzyme, which reacts with the substrate (e.g., the chromogenic reagent) to produce a protective barrier. For example, the second monoclonal antibody may be biotinylated to facilitate binding of enzyme-labeled streptavidin, where the enzyme produces an insoluble chemical material. Kits may also include the chromogenic reagent, e.g., a chromophoric substance including but not limited to DAB and Fast Red, which is processed by the second antibody. In other aspects, the probe may additionally comprise a first monoclonal antibody or other antigen binding fragment, specific to a molecule present in the biological sample.

In some aspects, it may be desirable to inactivate proteinase K. The kit may include a neutralization solution comprising a protease inhibitor such as PMSF or AEBSF (Pefabloc®) to permanently inactivate proteinase K. In some embodiments, the kit may include a processing step of heat (e.g., 95° C. for about 10 minutes) to inactivate proteinase K.

In other embodiments, a kit may contain one or more of the following reagents for immunostaining and microprotection, including:

Reagent 1: Non-Aqueous Mounting Medium (e.g., Xylene)

Reagent 2: Serum Blocking Reagent

Reagent 3: Primary Antibody Diluent Reagent (may comprise BSA)

Reagent 4: Biotinylated Secondary Antibody

Reagent 5: Streptavidin-Enzyme Conjugate

Reagent 6: Chromogen

Reagent 7: Chromogen Buffer (e.g., may contain other reagents such as 0.1% hydrogen peroxide)

Reagent 8: Chromogen Neutralization Solution (e.g., up to 30% hydrogen peroxide)

Reagent 9: Counter stain (e.g., hematoxylin)

Reagent 10: Microprotection Solution

Reagent 11: Microprotection Neutralization Solution

Reagents may be provided in concentrated form. In some embodiments, a primary mAb may be provided with one or more of Reagents 1-11. In other embodiments, a primary mAb may be provided with one or more of Reagents 2-8.

The HMP kit for biomolecule analysis (e.g., including analysis for DNA, RNA, lipid, protein, metabolite, etc.) from cells obtained from clinical or animal model tissue specimens will at least comprise a micropurification solution. Depending upon the usage for the particular type of kit, additional reagents may be provided to facilitate analysis of a particular biological sample, e.g., mRNA, miRNA, and proteomic kits, which may or may not be optimized according to the techniques provided herein. Solutions and protocols optimized for each biomolecule and application are provided herein.

In other aspects, the HMP kit can be used with commercial HC kits. For instance, IHC may be carried out using a commercial kit (e.g., http://zyagen.com/userfiles/Zyagen%20Peroxidase%20IHC%20Kits.pdf), and once staining is complete, the micropurification solution may be added to capture target and non-target cells.

### TaqMan™ Assays

Once isolated, the target cells may be subject to further analysis, e.g., DNA analysis or gene expression assays, such as a TaqMan® assay. A variety of TaqMan® gene expression assays are available (e.g., ThermoFisher's TaqMan® Gene Expression Assay, http://www.thermofisher.com/us/en/home/life-science/per/real-time-per/real-time-per-assays/taqman-gene-expression.html). Nucleotide hybridization probes may be obtained that specifically hybridize to DNA comprising the mutation of interest. In other aspects, custom hybridization probes may be designed in order to screen for particular mutations.

### Advantages

In addition to being widely accessible due to its simplicity and low cost, the methods described herein provide a significant improvement in precision compared to current methods. While laser dissection instruments have been used to perform ultra-precise procurement in the past, e.g., recovering individual nuclei, this is still an extremely challenging undertaking that typically retrieves only a few targets due to the tediousness of the human-based targeting process. For most downstream molecular assays, procuring a large number of cells or organelles is advantageous [23, 35, 36, 47-49], since the reproducibility and robustness of data sets generally improve as the amount of starting material increases.

Present techniques allow for moderate- and low-abundant molecules (miRNAs, mRNAs) and DNA allele variants to be isolated and even measured. This is important for assays such as proteomic assays wherein the dynamic range of expression is many orders of magnitude. Because micropurification is based on a molecular probe for targeting, all stained cells, organelles, or nuclei are rapidly retrieved when the histological slide is processed, whether that is one cell/organelle/nuclei or one million cells/organelles/nuclei. Thus, relatively large amounts of DNA, RNA or protein from individual cells or from subcellular structures can be recovered for molecular analysis using the micropurification process.

Micropurification may be used to select and purify target and non-target cells, organelles and nuclei, as well as other structures, facilitating further analysis of a variety of different cell types and biological molecules including DNA, RNA, proteins, lipids, metabolites, etc. The protocols and components/reagents may be tuned and optimized to isolate a wide variety of different types of cells (e.g., normal and diseased), and biological molecules. Such techniques also may be used to quantitatively validate the robustness and reproducibility of mutation testing, and to extend measurement capability to labile components such as mRNA and miRNA.

The methods and kits herein provide for a rapid, easy-to-use, precise (at a cellular level) and inexpensive cell procurement system that facilitates tumor mutation analysis for patient care and personalized medicine. These methods also are suitable for laboratory-based research, and do not rely upon sophisticated instrumentation, such as that found with laser dissection instruments. Accordingly, the methods may readily be performed in a variety of settings, including research labs, field applications, remote medical locations, etc.

In other aspects, the methods provided herein may be used to test for the presence of a variety of different types of tumor mutations. In some aspects, a series of histology samples may each be screened for the presence of a particular tumor marker. In other embodiments, cancer cells may be isolated, and the corresponding genetic material subjected to NGS to determine a mutation profile for a particular patient having a particular type of cancer.

In some aspects, processing may occur in parallel to facilitate high throughput analysis. Thus, in some aspects, a plurality of slides may be individually screened for the presence of a mutation or a protein in a manner of minutes (e.g., in about 10-20 minutes or more). The isolated cells having the protein or mutation may undergo further processing.

In some aspects, the methods provided herein may be performed manually. In other aspects, the methods may be incorporated into an automated system for processing histology slides, as human intervention or complex instrumentation is not needed to isolate target cells.

The HMP methods provided herein may be combined with microdissection, genomic analysis, and proteomic analysis. Such techniques can be used to facilitate personalized medicine, e.g., to quickly isolate a type of patient cell having a particular mutation or expressing a particular protein, wherein the isolated cells can undergo additional types of analysis, e.g., NGS, expression analysis, expression profiling, proteomic analysis, etc., to generate a personalized profile for a particular patient.

Other embodiments tailored to RNA studies may be developed using the techniques provided herein. While antigen retrieval may make non-target cells more susceptible to complete digestion and removal from the slide, this process may also hydrolyze RNA of both the target and non-target cells. As an alternative method, the micropurification solution may comprise RNase as the degrading enzyme to digest RNA in the non-target cells. For example, the protective barrier will prevent RNase from degrading RNA of protected cells. In another embodiment, if the RNase is able to penetrate the protective barrier, the RNase may be modified, e.g., to be tethered to a polymer to physically block passage through the barrier. The protected cells may then be collected, and their respective RNA obtained for further analysis. In this example, DNA, proteins, or other materials present in the non-target cells do not need to be removed (i.e., complete digestion is not needed) since the downstream molecular assay for RNA analysis is specific to RNA.

Specific examples of HMP protocols are provided below. These examples are not inclusive of all HMP embodiments and are intended only to show the practicality of the invention. While the present invention has been described in terms of particular embodiments and applications, it is not intended that these descriptions in any way limit its scope to any such embodiments and applications, and it will be understood that many substitutions, changes and variations in the described embodiments, applications, and details of the method and system illustrated herein and of their operation can be made by those skilled in the art without departing from the spirit of this invention.

In some embodiments, the digestion/degradation step may be omitted, i.e. placing the slide in the micropurification buffer, as it may not be desirable to digest/degrade the non-target cells. In such applications, the protective material may act as a barrier or inhibitor to protect the labeled cells from any of: a toxin to kill live cells, an endonuclease or DNase to degrade DNA, a RNase to degrade mi/mRNA, tyramide labeling, other proteinases, chemical cleavage of proteins, a lipase to degrade fat, etc. Thus, the present techniques may be used to provide a protective barrier for any of a variety of conditions.

In general, it is to be appreciated that the methods and techniques presented herein are not limited to one specific set of experimental conditions, e.g., with regard to heating times, cooling times, buffers, reagent concentrations, other processing conditions, and may include a range of conditions and reagents suitable for achieving the desired effect of microprotection of target cells and purification/analysis. All such conditions and reagents are considered to be within the scope of the present application.

### EXAMPLES

### Example 1: Methods of Micropurification of Target Cells

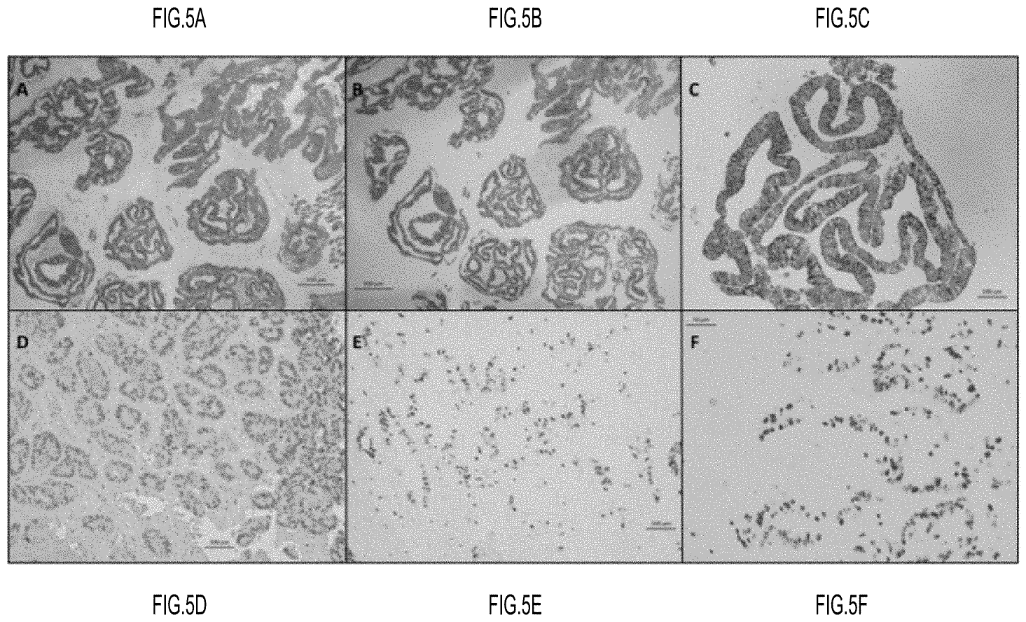

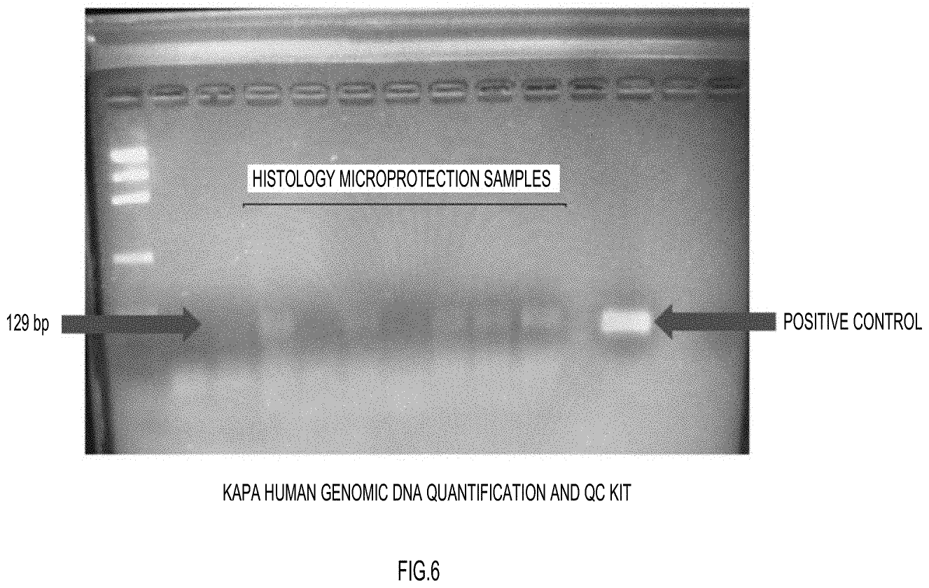

Example protocols for analyzing target cells are provided herein. This example also relates to FIGS. 5A-5F and FIG. 6. An immunohistochemical staining procedure is provided herein, which protects a target cell population from a micropurification solution, but does not protect unstained non-target cells. Enzymatic treatment of the stained tissue section digests the non-target cells into the micropurification solution (digestion buffer). The slide may be washed, and the target cells retrieved from the slide for molecular analysis.

Preparation

In some cases, ME stains were initially performed on tissue sections to assess morphology and cellular content. The microtissue slide was placed in xylenes for about 10 minutes, and was rehydrated using graded alcohols and water. Slides were placed in a staining rack within a Tissue Tek staining dish, filled with 250 ml of 1× Citra Plus buffer (BioGenex), and covered loosely.

Antigen Retrieval

After preparation, antigen retrieval was performed. Antigen retrieval involves exposing the surface of the cells, e.g., by melting polymer that may cover the cells during paraffin embedding and fixation of the biological sample onto the histology slide. Once exposed, antibodies or other molecular probes may contact the biological sample, e.g., to bind to markers, proteins, receptors, nucleotide sequences, etc. For slides embedded in paraffin, antigen retrieval may help induce or enhance susceptibility of the sample to micropurification.

The slides, covered by Citra Plus buffer, were microwaved for 5 minutes at high power (Sanyo, 1200 Watts) until boiling. The buffer level was checked and diH2O was added, as needed, to keep the tissue sections moist throughout the procedure. The slides were then microwaved for 15 minutes at power level 3, the buffer level was inspected halfway through the 15 minutes, and diH2O was added as needed. The slides were allowed to cool in the microwave for an additional 20 minutes. Finally, the slides were rinsed with diH2O.

Labeling

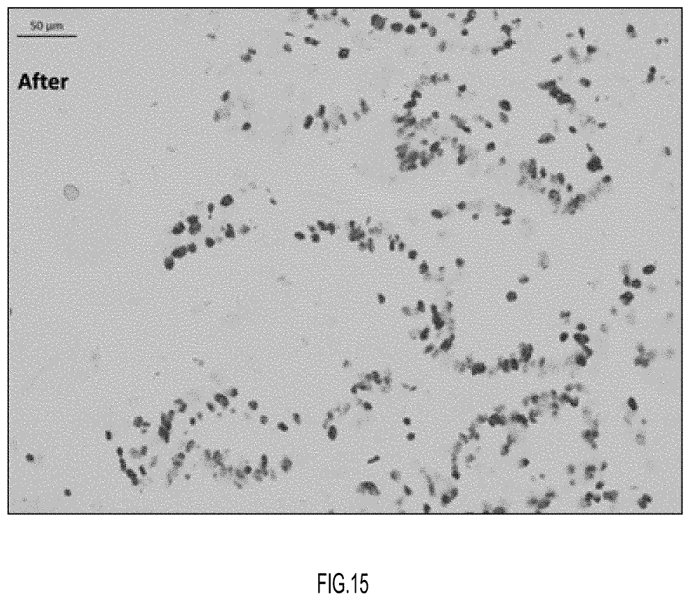

Following antigen retrieval, the slides were incubated at room temperature for about two hours with primary antibody, for about one hour with secondary antibody, and then detection was completed using DAB chromogen with no counterstain. The primary antibodies used for immunohistochemistry were cytokeratin AE1/AE3 mAbs (1:100, BioGenex) and anti-Ki-67 mAbs (1:100, BioGenex) (see, e.g., FIGS. 5A-5F, FIGS. 14A-14B, and 15). After DAB deposition, slides were rinsed in water and imaged using an AxioLab.A1 microscope and Axiocam 105 camera.

Microprotection

Slides were incubated in 200-300 microliters of micropurification solution (also referred to as digestion buffer) containing 1 mg/ml proteinase K and 0.1% SDS for 30 mins at 56° C. The digestion step dissolved away the non-microprotected cells and extracellular structures the non-stained cells and structures). The micropurification solution was decanted, and the slides were washed three times in PBS and imaged, followed by scraping of the microprotected cells that remained on the slide into a fresh PBS buffer solution for molecular analysis. The target cells, recovered from the slide by manual scraping, were analyzed, demonstrating that the DNA in the target cells is present and amenable to PCR amplification (see, e.g., FIG. 6).

In some cases, immunohistochemical staining was performed before and after HMP. FIGS. 5A-5F show a series of microscopic images for histological sections of multiple samples of colorectal cancer before (see, e.g., FIGS. 5A and 5D) and after (see, e.g., 5B, 5C, 5E and 5F) HMP. Immunohistochemical staining of a histology section from several patients having colorectal cancer produced a dark DAB precipitate over the tumor cells (labeled with an anti-cytokeratin mAb) or over the tumor cell nuclei (labeled with an anti Ki 67 mAb) as shown in FIG. 5A-5F. Subsequent incubation of the histological section with a buffer containing proteinase K and SDS digested the non-target cells off the slide but left the targeted tumor cells intact, in effect, separating the tumor cells from the non-tumor cells.

Molecular Analysis of DNA

DNA was extracted from the recovered cells via overnight incubation with proteinase K at 65° C. with shaking (500 rpm). Samples were then heated at 95° C. for 15 minutes to inactivate proteinase K. PCR with the extracted DNA samples was performed with the KAPA Human Genomic DNA Quantification and QC Kit for the 129 bp fragment. Forty PCR cycles were completed with each cycle at 95° C. for 10 seconds and 62° C. for 30 seconds. Amplified DNA samples were run on a 2% TBE agarose gel with ethidium bromide staining. The gel was imaged on a UVP ChemiDoc-IT.

Similar techniques may be employed to recover and analyze additional biomolecules in the target cells)/structure, including RNA, proteins, lipids, metabolites, and others. For example, the isolated cells may be subjected to RNA analysis, e.g., using an RNeasy kit from Qiagen to obtain RNA, In other aspects, the isolated cells may be subjected to protein analysis, e.g., using a ReadyPrep protein extraction kit to capture proteins from Bio-Rad. In still other aspects, Abcam's lipid extraction kit may be used to isolate lipids. Accordingly, once isolated, the cells may be subjected to a variety of different types of analysis to recover and analyze biomolecules in the target cell(s)/structure, including RNA, proteins, lipids, metabolites, and so forth.

### Example 2: Methods of Purification of Non-Target Cells

Example protocols for analyzing non-target cells are provided herein, An immunohistochemical staining procedure is provided herein, which protects a target cell population from a micropurification solution, but does not protect unstained non-target cells. Enzymatic treatment of the stained tissue section digests the non-target cells into the micropurification solution (digestion buffer) and allows the non-target cells to be collected for molecular analysis.

Preparation

In some cases, H&E stains were performed on tissue samples to first assess morphology and cellular content. Immunohistochemistry staining was completed after placing the slide in xylenes for 10 minutes and rehydrating the slides using graded alcohols and water, prior to antigen retrieval.

Slides were placed in a staining rack within a Tissue Tek staining dish, filled with 250 ml of 1× Citra Plus buffer (BioGenex), and covered loosely.

Antigen Retrieval

The slides, covered by Citra Plus buffer, were microwaved for 5 minutes at high power (Sanyo, 1200 Watts) until boiling. The buffer level was checked and diH2O was added, as needed. The tissue sections were kept moist throughout the procedure. The slides were then microwaved for 15 minutes at power level 3 and the buffer level was inspected halfway through the 15 minutes and diH2O added as needed. The slides were allowed to cool in the microwave for an additional 20 minutes. Finally, the slides were rinsed with diH2O.

Labeling

Following antigen retrieval, the slides were incubated at room temperature for about, two hours with primary antibody, for about one hour with secondary antibody, and then detection was completed using the iView kit and DAB chromogen with no counterstain. The primary antibodies used for immunohistochemistry were cytokeratin AE1/AE3 mAbs (1:100, BioGenex) and anti-Ki67 mAbs (1:100, BioGenex). After DAB deposition, slides were rinsed in water and imaged using an AxioLab.A1 microscope and Axiocam 105 camera.

Microprotection

Slides were incubated in 200-300 microliters of micropurification solution (digestion buffer) containing 1 mg/ml proteinase K and 0.1% SDS for 30 minutes at 56° C. The digestion step solubilized the non-microprotected cells and structures (i.e., the non-stained cells), which were collected in the digestion buffer for subsequent molecular analysis.

Molecular Analysis

Immunohistochemical staining of a histology section from several cases containing colorectal cancer produced a dark DAB precipitate over the tumor cells (cytokeratin) or over the tumor cell nuclei (Ki-67) as shown in FIGS. 5A-5F. Subsequent incubation of the histological section with the buffer containing proteinase K and SDS digested the non-target cells off the slide but left the targeted tumor cells intact, in effect separating the tumor cells away from the non-tumor cells. The digested cells/structures were recovered from the slide in the digestion buffer for subsequent analysis.

### Example 3. Optimization of Cell and Nuclear Enrichment

Mixtures of cultured cells with known genomic status are prepared to: a) quantitatively assess the efficiency of target cell enrichment of cells and nuclei; and b) optimize the HMP process by varying one or more of the purification components, temperature, time, slide preparation, and IHC conditions in order to determine optimal conditions.

Cell Block and Slide Preparation

Human tumor cell lines, such as Burkitt lymphoma ST486 (KRAS and BRAF wild-type), lung carcinoma A549 (KRAS+ homozygous mutation), and melanoma UACC.62 (BRAF+ heterozygous mutation) are obtained from the ATCC. Cells are grown to near confluence in T-125 flasks and recovered into PBS buffer. ST486 cells are admixed with A549 or UACC.62 at 1:100, 1:10, 50:50, 10:1, and 100:1 ratios. Each admixed sample is processed into a formalin-fixed, paraffin-embedded cellblock. Histological sections from each block are sliced at 4-, 10-, and 25-micron thickness. Sections are unbaked or baked at 60° C. for 20 minutes, dewaxed, and rehydrated through xylenes and graded alcohols, using a short (about 2 min per step) or long (about 10 min per step) protocol. Triplicate recut sections are prepared from each block.

Antigen Retrieval

Slides are placed in a staining rack in a Tissue Tek staining dish filled with 250 ml of 1× Citra Plus buffer (BioGenex). The slides are microwaved for 5 minutes at maximal power (Sanyo, 1200 Watts) until boiling, are microwaved for an additional 15 minutes at medium power, then are cooled for 20 minutes and rinsed with diH2O.

In parallel, additional slides are processed through a range of heating times, temperatures, and buffer conditions.

Labeling

Slides are incubated at room temperature for 30 minutes with a primary antibody (using a range of titers), for 30 minutes with a secondary antibody, and then detected using DAB or Fast Red or another suitable chromogen. Select slides are counterstained using IHC (e.g., with H&E) and rinsed in water.

Slides may be incubated at or about room temperature for any amount of time ranging from 1 to 60 minutes for the primary antibody, and from 10 to 60 minutes with the secondary antibody.

For example, cell targeting antibodies may bind to cytokeratin, melanoma antigen recognized by T cells (MART-1), or transcription termination factor 1 (TTF1) while nuclear targeting antibodies bind to Ki-67 or histone, each of which has been successfully performed in previous studies [50].

Micropurification

Slides are incubated in 200-300 microliters of micropurification solution for about 10 mins at 56° C. The incubation time may vary anywhere from 1 minute to 30 minutes, or even 60 minutes. Conditions to be assessed include varying the major components of the purification solution (e.g., detergents, proteases, additional enzymes, buffers, and pH, etc.) by varying the amounts of one component, two components, three components, four components, five components, and so forth during each processing run, as well as by varying the time and temperature of the processing run.

Non-stained cells and structures are recovered for downstream molecular analysis. The slides are washed and imaged. After imaging, the stained target cells/structures are retrieved for subsequent analysis.

Imaging

Digital imaging of the histological sections may be performed to quantitate procurement efficiency using an AxioLab.A1 microscope and Axiocam 105 camera. The purity of the isolated cells and nuclei are assessed by both manual and auto-counting. Triplicate serial recut sections are evaluated for reproducibility.

DNA Analysis

DNA is extracted from the recovered cells and nuclei after overnight incubation with proteinase K at 65° C. with shaking (500 rpm). Samples are heated at 95° C. for 15 minutes to inactivate proteinase K.

Quantitative PCR (qPCR) with the extracted DNA is performed using the KAPA Human Genomic DNA Quantification and QC Kit for the 305 bp, 129 bp and 41 bp fragments. Forty PCR cycles are run with each cycle at 95° C. for 10 seconds and 62° C. for 30 seconds. All assays are completed in triplicate. The Q129/Q41 and Q305/Q41 ratios are calculated to determine DNA quality.

For cell enrichment, Taqman qPCR may be used to measure the mutant and wild type allele ratio in each cell admixture sample (KRAS and BRAF wild type; KRAS+ mutation, BRAF+ mutation). For nuclear enrichment, Taqman qPCR is used to measure genomic DNA and mitochondrial DNA in whole cells versus purified nuclei, and the ratio is assessed for each set of micropurification conditions [51-54].

To measure quality of the assay, DNA KAPA Q-ratios may be generated which indicate the quality of the recovered DNA. Target cells and nuclei are procured with >95% purity and >95% efficiency based on analysis of the digital images of the histology slides. The purity of target cells and nuclei is >95% based on analysis of the DNA mutation data and the ratios of genomic to mitochondrial DNA, respectively. Slide-to-slide reproducibility for cellular and nuclear enrichment is >95%. Thus, the quality is sufficient for standard DNA allelotyping and sequencing assays that are routinely performed in the clinic and research laboratory, and can be carried forward to future R&D and commercialization efforts.

Protocols similar to the micropurification protocol used for KRAS gene mutation analyses may be used to analyze additional types of tumor mutations in cells.

By optimizing the purification process, as described herein, the cells may be purified to a level sufficient to meet the proficiency standards of a clinical assay in a CLIA laboratory.

### Example 4. mRNA and miRNA Analyses

The status of mRNA and miRNA after micropurification and optimization is evaluated to determine whether quantitative reverse transcriptase PCR (qRT-PCR) analysis can be performed.

Cell Block and Slide Preparation

Anonymized human tumor specimens are obtained from the Department of Pathology at Sinai Hospital, Baltimore, Md. Histological sections from each block are sliced at 4-, 10-, and 25-micron thickness. Sections are unbaked or baked at 60° C. for 20 minutes, dewaxed, and rehydrated through xylenes and graded alcohols, using a short (about 2 min per step), intermediate (about 5 min per step), or long (about 10-15 min per step) protocol. Triplicate recut histological sections are prepared from each block.

Antigen Retrieval

Slides are placed in a staining rack within a Tissue Tek staining dish, filled with 250 ml of 1× Citra Plus buffer (BioGenex), and covered loosely. One set of slides is microwaved using standard conditions comprising: microwaving the slides for 5 minutes at maximal power (Sanyo, 1200 Watts) until boiling, microwaving the slides for an additional 15 minutes at medium power, and then cooling the slides for about 20 minutes and rinsing with diH2O.

In parallel, additional slides are processed through a range of heating times, temperatures, and buffer conditions. To identify optimized conditions, parameters (e.g., processing times, concentrations, temperatures, etc.) may be varied 10× higher and 10× lower from condition(s) that produce the desired effect, e.g., microprotection of target cells under conditions that do not lead to substantial degradation of the underlying biological material to be analyzed (e.g., by DNA analysis, RNA analysis, protein analysis, lipid analysis, etc.).

Labeling

Slides are incubated at room temperature for 30 minutes with primary antibody, for 30 minutes with secondary antibody, and are then stained with DAB or Fast Red chromogen with no counterstain. After DAB deposition, the slides are rinsed in water and imaged using an AxioLab.A1 microscope and Axiocam 105 camera to document IHC staining quality.

Slides may be incubated at or about room temperature for any amount of time ranging from 1 to 60 minutes for the primary antibody, and from 10 to 60 minutes with the secondary antibody.

Micropurification

Slides are incubated in 200-300 microliters of a purification solution for 10 mins at 56° C. The incubation time may vary anywhere from 1 minute to 30 minutes, or even 60 minutes. In some aspects, a 1 mg/mL proteinase K in Tris buffer with 1% SDS is used as the micropurification buffer. Conditions may be varied, and therefore, conditions to be assessed include all of the major components of the purification solution (e.g., detergents, proteases, additional enzymes, buffers, pH, etc.) by varying the amounts of one component, two components, three components, four components, five components, and so forth during each processing run, as well as by varying the time and temperature of the processing run.

Non-stained cells and structures are recovered for downstream molecular analysis. The slides are washed and imaged. After imaging, the stained target cells/structures are retrieved for subsequent analysis.

The process is repeated on three recut histological sections to assess reproducibility, and the slides are imaged using an AxioLab.A1 microscope and Axiocam 105 camera to document the status of micropurification.

mRNA and miRNA Analysis

mRNA and/or miRNA are extracted from the micropurified cells samples using Qiagen RNeasy® and Qiagen miRNeasy FFPE® kits, respectively, according to the manufacturer's instructions.

Initial quality and quantity assessment of the extracted RNA is determined with a Qubit RNA IQ assay using a Qubit 4 Fluorimeter (ThermoFisher Scientific). For quantity assessment of miRNA samples, the Qubit microRNA Assay Kit is used. The extracted RNA is converted to cDNA using a High Capacity cDNA Reverse Transcription Kit (ThermoFisher Scientific). 10 ng of RNA is used for each reaction. The thermal cycler is set for the following: 25° C. for 10 minutes, 37° C. for 120 minutes, 85° C. for 5 minutes.

qRT-PCR is performed on a StepOnePlus instrument (ThermoFisher Scientific). Two human endogenous controls [Human ACTB (Beta Actin) and Human GAPD (GAPDH)] are used to assess RNA levels across samples. Forty PCR cycles are run with each cycle at 95° C. for 1 second and 60° C. for 20 seconds. All assays are completed in triplicate. For miRNA analysis, the TaqMan Advanced miRNA Human Endogenous Control 96-well plate is used. cDNA template is generated according to the manufacturer's instructions prior to running the assay.

All micropurified samples are compared to macrodissected (i.e. scraped) immunostained serial recut tissue sections to evaluate the impact of the micropurification process on RNA/miRNA quality and quantity.

In some embodiments, biomolecule hydrolysis may occur during antigen retrieval or IHC staining, for components such as mRNA/miRNA that may be more labile. The protocol (e.g., antigen retrieval and/or IHC staining) may be adjusted to reduce or eliminate hydrolysis.

Thus, the tunability of the methods presented herein is a particular strength of micropurification. Multiple options are available (e.g., one could use different reagents for the labels or for the micropurification solution, or one could use different experimental conditions for the micropurification process (e.g., different temperatures, duration of processing steps, etc.) for completing the micropurification process. Accordingly, the protocol may be adjusted to avoid nucleic acid hydrolysis, if needed.

Detection of Beta-Actin and GAPDH Transcripts

Detection of beta-actin and GAPDH transcripts in the 20-30 Ct level is achieved using qRT-PCR after the micropurification process. Detection of miRNA endogenous control panel genes in the 20-30 Ct level is achieved after the micropurification process. A range of values is expected depending on the abundance of each miRNA in the target cells and subcellular structures, and thus, the level of each miRNA is carefully compared between the purified samples and the tissue scrape controls to ensure all expressed miRNAs are successfully measured using the new technology.

### Example 5: Micropurification Kit for Cells

Micropurification kits may be provided to isolate specific types of cells without using complex instrumentation. For example, a micropurification kit may allow rapid and inexpensive procurement of specific types of cells using the techniques provided herein. Once the cells have been isolated, and thus, purified from the biological sample (e.g., from clinical or animal model tissue specimens affixed to a histological slide), the isolated cells may be subjected to further analysis, e.g., DNA analysis, RNA analysis (including mRNA, miRNA, etc.), proteomic analysis, etc. Such analyses may be used to build a profile of the specific type of isolated cell. For example, such kits may facilitate tumor mutation analysis for patient care and personalized medicine. For each type of kit, solutions and protocols may be optimized for each biomolecule type and application.

In some aspects, kits may utilize an immunohistochemical staining procedure to label a target cell population with an insoluble chemical material that protects the labeled and stained cells. Subsequent treatment with a micropurification solution, which includes one or more enzymes which digest the non-stained cells of the tissue section, allows for collection and molecular analysis of target cell contents.

### Example 6: Micropurification of Clinical Samples Using DAB

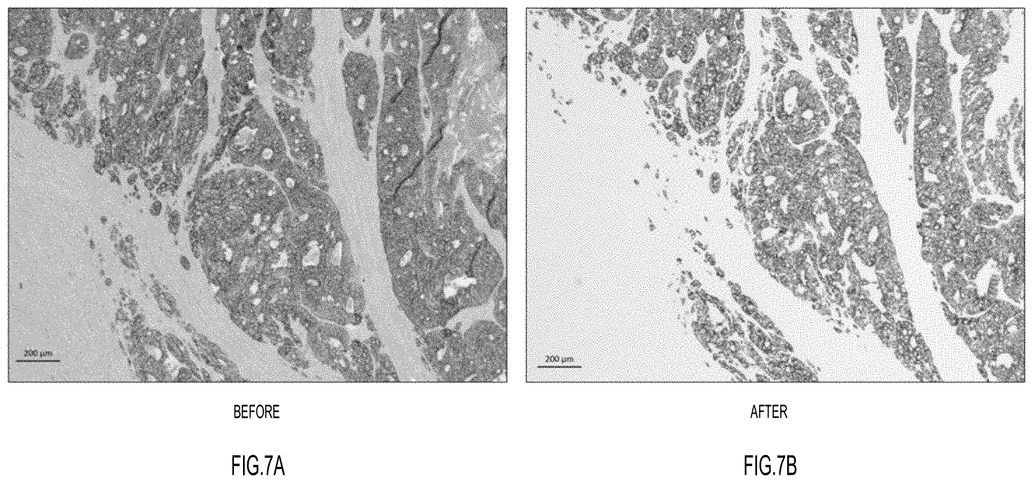

The micropurification process was performed on clinical samples. FIGS. 7A-9B show invasive colorectal cancer and lymphocytes before and after immunohistochemical (IHC) staining.

FIGS. 7A and 7B are two serial histological sections of colon carcinoma both stained with cytokeratin AE1/AE3 mAb antibodies followed by deposition of 3,3′-diamino benzidine (DAB) chromogen (at a 50× magnification) onto the labeled cells. FIG. 7A was also counterstained with hematoxylin and eosin (H&E) to allow visualization of the nontarget cells/structures (stroma, fibroblasts, inflammatory cells, nerves), which appeared as pink or red, and are shown as lightly stained in the grayscale image. Thus, FIG. 7A shows the histological section before micropurification solution.

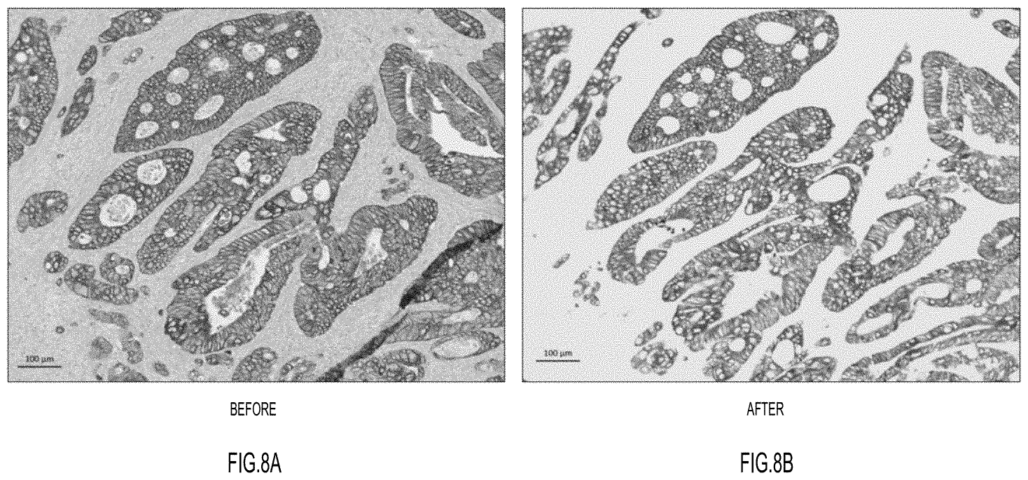

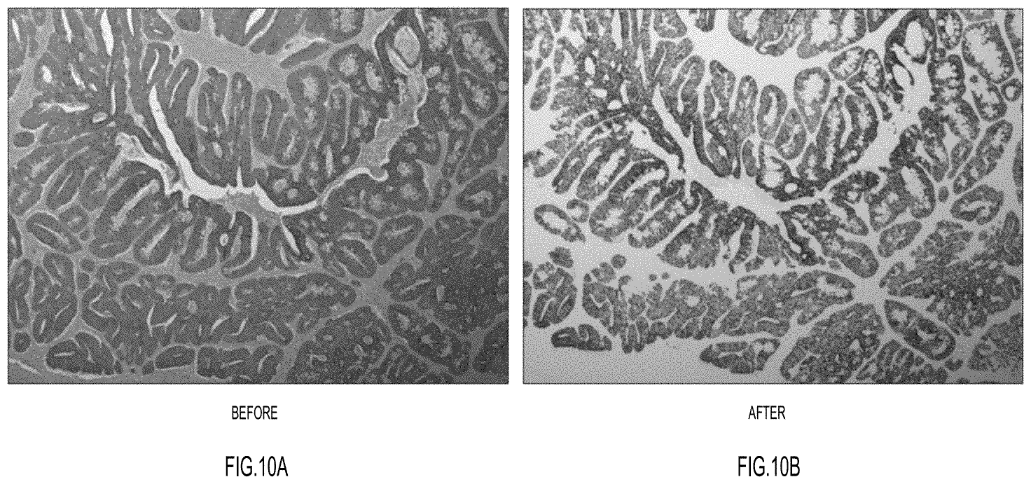

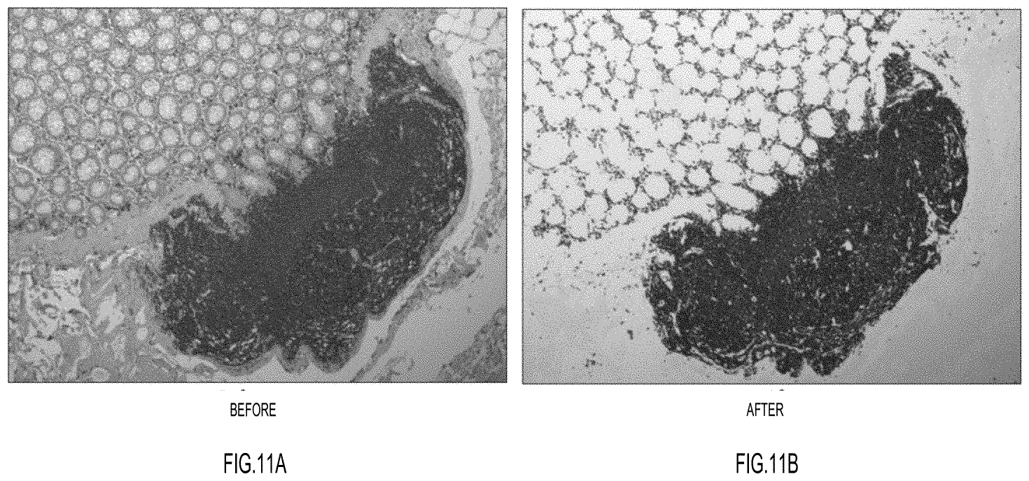

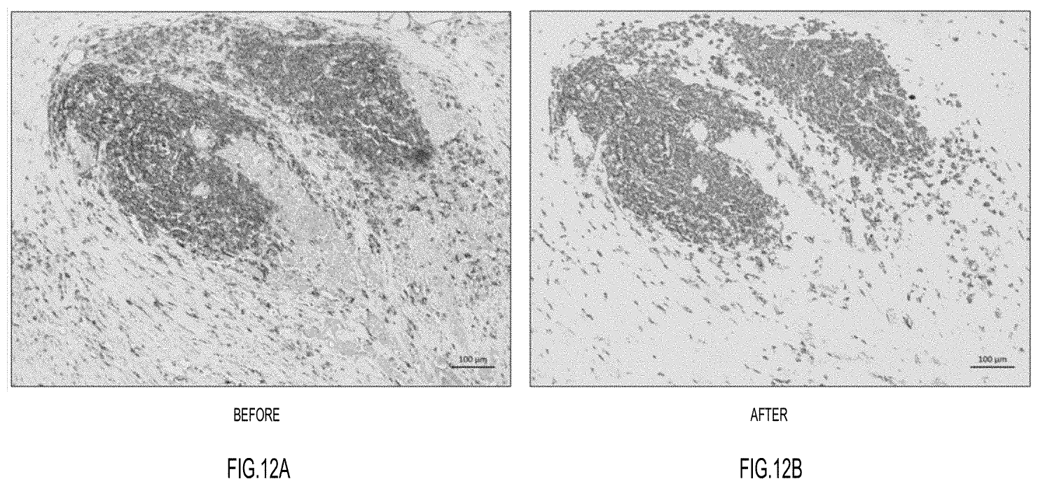

FIG. 7B was subjected to micropurification and similarly counterstained with H&E. The target cells remain intact on the slide but the non-targets are solubilized and no longer are visible. Thus, FIG. 7B shows the histological section after the micropurification solution was added, as the non-target cells are no longer present. In both FIGS. 7A and 7B, the stained cancer cells were darkly colored (shown as darkly stained in the grayscale image). Similar experiments have been performed on a variety of histological sections, as shown in FIGS. 8A-14B.

High-power microscopic visualization has been used to verify that the process strips the non-stained areas down to glass with no remaining cells, organelles, or other structures. For example, FIGS. 8A and 8B show a similar colon carcinoma stained with the insoluble chemical material before and after purification at 100× magnification.

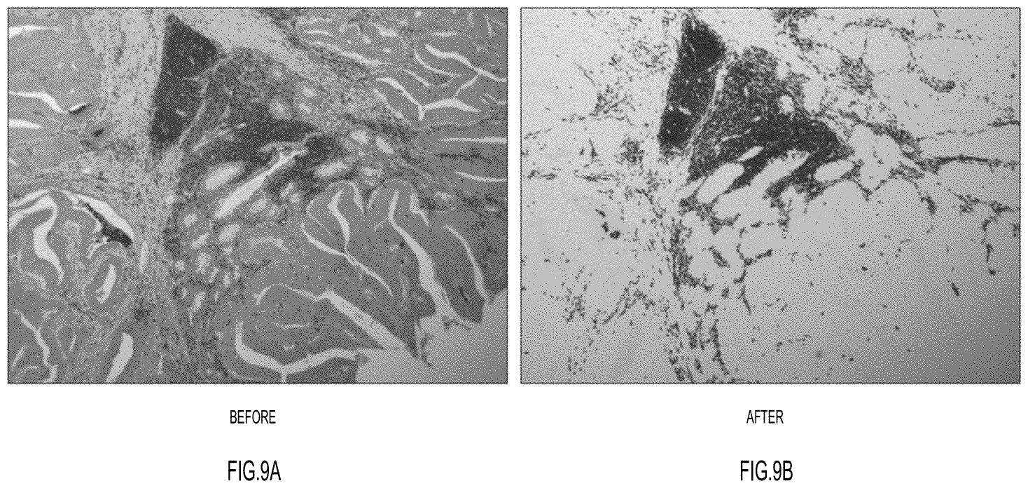

FIGS. 9A and 9B show images of a colon carcinoma with areas of significant chronic inflammation as shown by staining with a panlymphocyte antibody cocktail against CD8, CD20, and CD60 (50× magnification), which are inflammation markers. The lymphocyte population consisted of foci of intense inflammation in the upper middle part of the slide, with thin bands of lymphocytes streaming through the stroma, and scattered individual lymphocytes elsewhere throughout the section (FIG. 9A). After the micropurification process, a pure population of lymphocytes remained on the slide, including the inflammatory foci, lymphocyte bands, and individual cells (FIG. 9B).

### Example 7: Micropurification of Clinical Samples Using Fast Red

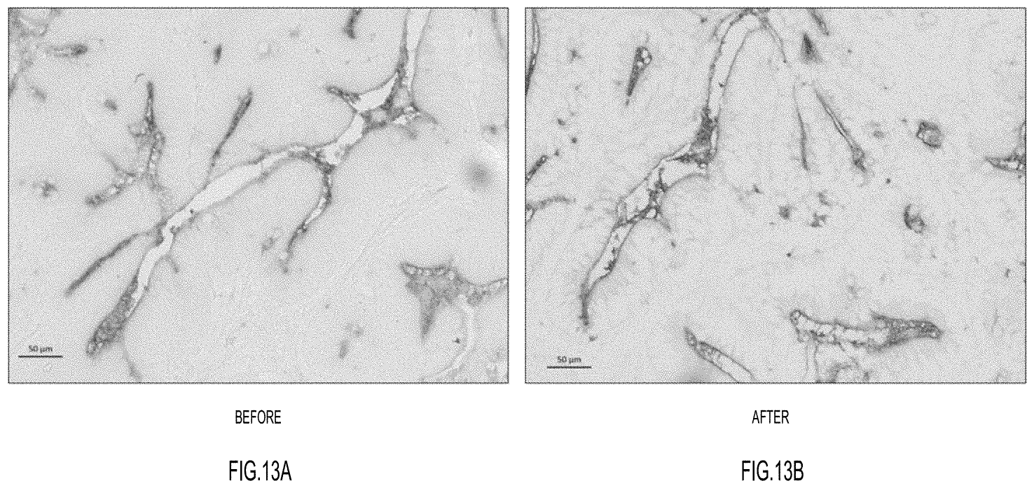

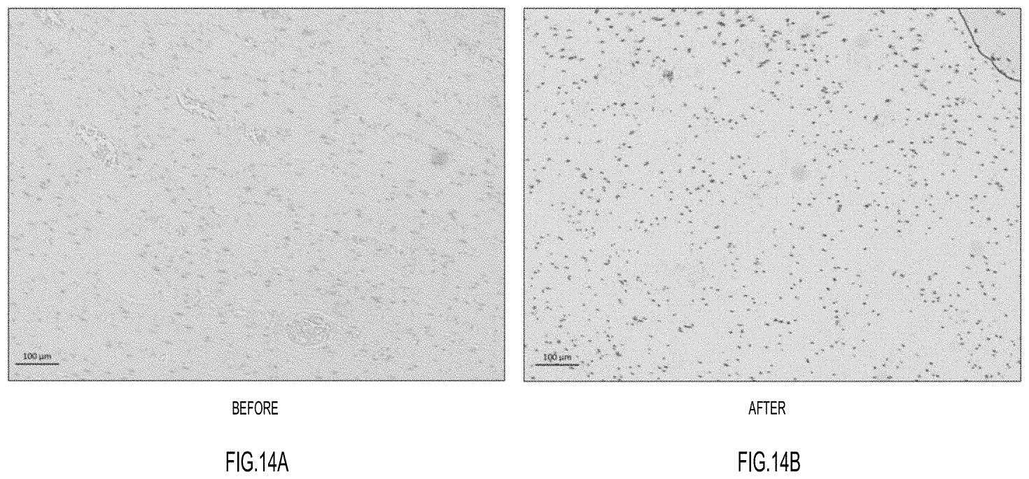

As another example, involving purification of membranes, colon cancer was stained with an antibody against epithelial membrane antigen (EMA), using Fast Red as the chromogen instead of DAB (FIG. 13A-14B). The tumor cells in FIG. 13A had a light pink blush covering the cytoplasm, with strong membrane staining associated with luminal structures. After micropurification, the non-stained tumor cell and extracellular matrix components were completely removed leaving only the stained membranes (FIG. 13B). Strong lumen-adjacent staining was observable, but also seen under high-power magnification were light spindly cellular membranes that were stained and present throughout the section.

Nuclear procurement was demonstrated in FIGS. 14A and 14B that show colon carcinoma immunostained with an antibody against Ki-67 nuclear protein before and after micropurification. Pink target tumor nuclei were seen on the slide in FIG. 14A (shown as medium gray dots on the grayscale image) and the tumor cell cytoplasm had a light pink blush. After purification, all cytoplasm was removed leaving behind distinctly appearing nuclei (shown as dark gray dots on the grayscale image) on the slide (FIG. 14B).

### Example 8: KRAS Gene Mutation Analysis Using HMP

To assess target cell biomolecules after micropurification, three clinical cases of colorectal cancer with a known mutation in the KRAS gene were investigated. KRAS has been implicated in a variety of cancers, including colon and breast cancer. After the purification process, DNA in the tumor cells was recovered and subjected to TaqMan PCR analysis to measure mutant KRAS.

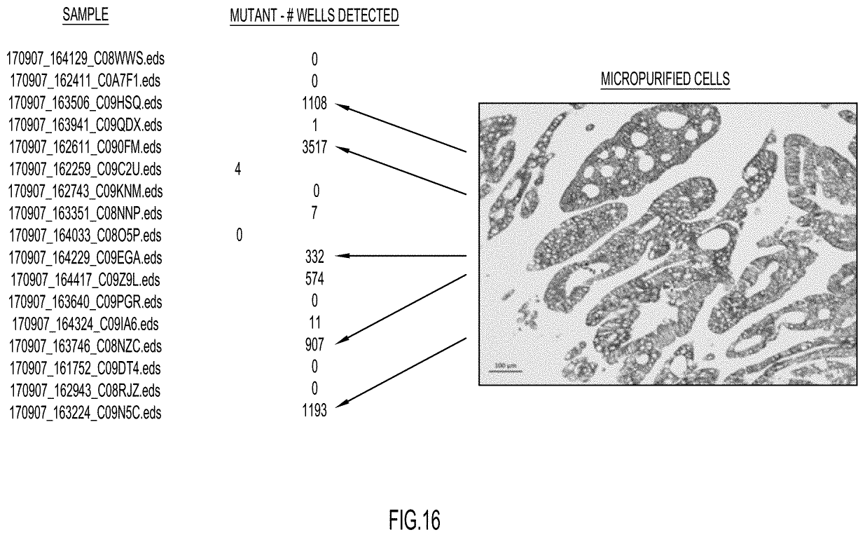

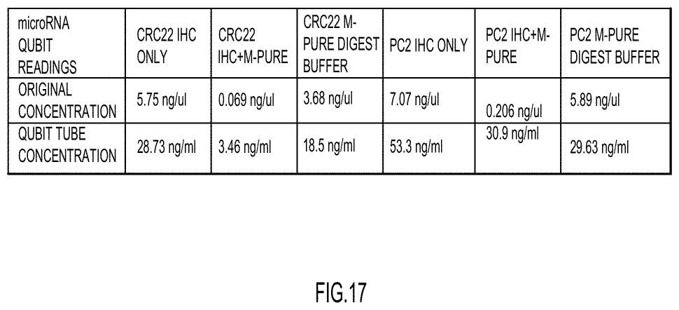

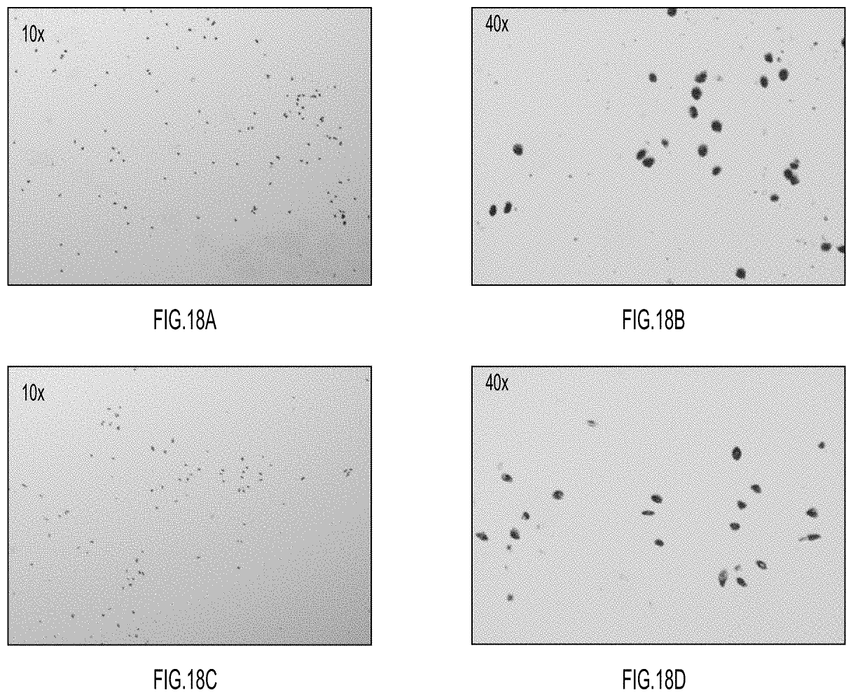

FIG. 16 shows data from a biological sample having a c.34G>A mutation (with corresponding amino acid sequence mutation p.G12S), and also shows that the mutant allele was detected above background in the six tumor containing samples (see arrows), but not in the control samples.

Similar results were observed in the additional two colon cancer cases with a KRAS gene mutation (not shown), wherein the mutations were: (a) nucleotide mutation c.35G>T (with corresponding amino acid sequence mutation p.G12V); (b) nucleotide mutation c.34G>A (with corresponding amino acid sequence mutation p.G12D). Overall, 48 micropurification samples (22 mutation positive and 26 controls) were analyzed and mutant KRAS was detected in all of the mutation positive samples but in none of the controls. Thus, the detection sensitivity was 100% and the false positive rate was zero. Accordingly, not only do the HMP assays provided herein allow for DNA recovery, but also, the recovered DNA may be sequenced to identify the presence of mutations.

In general, a variety of different types of gene mutations can be screened for using the techniques presented herein.

Successful detection of a genomic mutation in clinical samples demonstrates that the present techniques and systems are compatible with DNA allelotyping of patient and animal model tissues, as the DNA in the isolated target cells is intact and measurable.

### Example 9: General IHC Procedure

A general IHC procedure is provided as follows.
1. Dewax and rehydrate slides by placing in xylenes for 15 minutes (2×) and then in graded alcohols (100%, 95%, 70%) for 2 minutes (in each solution) in the hood.
2. Place slides in diH2O for 2 minutes (in hood), and make antigen retrieval dilution: 25 mL antigen retrieval solution (10× Citra Plus Buffer “Biogenex”) diluted in 225 mL of diH₂O.
3. (Antigen retrieval step) heat slides in diluted 1× Citra plus Buffer “Biogenex” (Step 2) for 5 minutes on 100% power in microwave then add diH₂O to fill tub up to the line (250 mL).
4. (Antigen retrieval step continued) Heat slides in 1× Citra plus Buffer for 15 minutes at 30% power in microwave (pause and add more diH₂O to fill tub up to the line once more).
5. Let slides cool in Citra Plus Buffer for 20 minutes on bench top.
6. Wash slides in new tub of 1×PBS (250 mL).
7. Outline tissue section with hydrophobic pap pen.
8. Apply peroxidase block (Dako or Invitrogen) for 10 minutes and room temperature (For each slide, apply pap pen outline and immediately add peroxidase block after so as not to let the slide dry. For each slide add 250 uL-300 uL depending on how large the tissue section is to cover the entire section outlined).
9. After 10 minutes, wash the slide(s) in a fresh tub of 1×PBS (250 mL).
10. Apply primary antibody for 30 minutes at room temperature (RT) (250 uL for each). Decant antibody solution off when finished and proceed to Step 11.
11. Wash slide(s) in a fresh tub of 1×PBS (250 mL).
12. Apply secondary antibody (DAKO Envision+ Anti-Mouse) for 30 minutes at RT (apply 2 to 3 drops to cover slide(s)).
13. Wash slides in fresh 1×PBS (250 mL).
14. Apply DAB solution (prepared and vortexed in a 1.5 mL tube: Dako=2 drops DAB chromogen+1 mL substrate buffer) for 5 minutes at RT.
15. Wash slides in diH₂O.
After IHC staining, the slide may be optionally counterstained or may be processed according to the micropurification protocol.

### Example 10: Counterstain Procedure

A general counterstain procedure is provided as follows. The slides are placed in each of the following solutions for 30 seconds each (in order).
a. Hematoxylin
b. diH₂O
c. Bluing agent
d. 70% EtOH
e. 95% EtOH
f. 95% EtOH
g. 100% EtOH

The slides are then placed in each of the following solutions for 2 minutes each (in order):
h. Xylenes
i. Xylenes

Thus, the slides are dehydrated through graded alcohol (70%, 95%, 100%) and xylenes for 2 minutes each in the hood. The slides are allowed to air-dry in the hood for at least ten minutes before covering with a coverslip.

### Example 11. Optimized Micropurification Procedure

A general micropurification procedure is provided as follows.
1. Prepare micropurification solution (also referred to as m-pure solution) (In Tris buffer, 0-1.0% concentration of SDS (Sigma Aldrich) and 0.1-1.0 mg/mL proteinase K) and set the heat block to 56° Celsius (C).
2. Wash slides briefly in Tris buffer and place slides one by one on the heating block.
3. Immediately re-circle tissue with pap pen and apply ˜250 μL of m-pure solution to each slide. Let sit for 10 minutes (after 5 minutes apply another 250 uL of m-pure solution to each slide).
4. After ten minutes wash in a 0.1% SDS solution in diH₂O (250 mL).
5. Wash in diH₂O and proceed to dewax and cover slip or proceed to DNA, RNA, protein, etc. purification.
It is noted that other suitable micropurification times may be suitable, e.g., ranging from 5 minutes to 30 minutes or any range in between. Additional buffer may be added to prevent slides from drying out.

### Example 12. DNA Isolation Using Micropurified Tissue Sections

A DNA isolation procedure using a Qiagen GeneRead DNA FFPE Kit is provided as follows.
1. Scrape the slide(s) with a scalpel/razor into a 1.5 mL microcentrifuge tube with 55 μl RNase-free water, 25 μl buffer FTB, and 20 μl proteinase K. Centrifuge so all contents are in the bottom of the tube.
2. Incubate overnight at 56° C. in the thermomixer at 400 rpm. (Thermomixer used: Eppendorf Thermomixer C, model #5382.)
3. Following the overnight step, incubate in the thermomixer at 400 rpm at 90° C. for one hour.
4. Briefly centrifuge tubes.
5. Add 115 μl RNase-free water and mix.
6. Add 35 μl UNG (Qiagen), vortex and incubate in thermomixer at 50° C. for 1 hour.
7. Briefly centrifuge to remove drops from lid.
8. Add 2 μl RNase A (100 mg/ml) mix, incubate for 2 minutes at RT.
9. Add 250 μl AL Buffer and mix with vortex.
10. Add 250 μl 100% ethanol and mix with vortex.
11. Centrifuge briefly.
12. Transfer 700 μl lysate to qIAamp MinElute column (in a 2 ml collection tube) and centrifuge at max speed for 1 min.
13. Discard flow-through and reuse collection tube. Repeat step 12 until all lysate is used.
14. Add 500 μl AW1 Buffer to spin column and centrifuge at max speed for 1 min. Discard flow through and reuse collection tube.
15. Add 500 μl AW2 Buffer to spin column and centrifuge at max speed for 1 min. Discard flow through and reuse collection tube.
16. Add 250 μl 100% ethanol to spin column and centrifuge at max speed for 1 min. Discard flow-through and collection tube.
17. Place spin column in new 2 mL collection tube and centrifuge at max speed for 1 min.
18. Discard the collection tube and place in newly labeled 1.5 mL microcentrifuge tube and add 30 μl ATE Buffer to the column.
19. Incubate at RT for 5 min and centrifuge at max speed for 1 min. Discard MinElute spin column and keep eluted DNA for further analysis. Store at −20° C. for long term storage.

### Example 13. RNA Isolation from Micropurified Tissue Sections

A RNA isolation procedure based on Qiagen's RNeasy FFPE Tissue Section kit is provided as follows.
1. Scrape slide(s) with a scalpel/razor into a 1.5 mL microcentrifuge tube with 150 μl PKD buffer.
2. Add 10 μl proteinase K. Mix gently by pipetting up and down.
3. Incubate at 56° C. for 15 min, then at 80° C. for 15 min.
4. Incubate on ice for 3 min. Then, centrifuge for 15 min at 20,000×g (13,500 rpm).
5. Transfer the supernatant to a new microcentrifuge tube taking care not to disturb the pellet.
6. Add DNase Booster Buffer equivalent to a tenth of the total sample volume (approximately 16 μl) and 10 μl DNase 1 stock solution. Mix by inverting the tube. Centrifuge briefly to collect residual liquid from the sides of the tube.
7. Incubate at RT for 15 min.
8. Add 720 μl ethanol (100%) to the sample, and mix well by pipetting. Do not centrifuge. Proceed immediately to step 9.
9. Transfer 700 μl of the sample, including any precipitates that may have formed, to an RNeasy MinElute spin column placed in a 2 ml collection tube. Close the lid gently, and centrifuge for 15 seconds at greater than or equal to 8000×g (greater than or equal to 10,000 rpm). Discard the flow through.
10. Repeat step 9 until the entire sample has passed through the RNeasy MinElute spin column.
11. Add 500 μl RPE Buffer to the RNeasy MinElute spin column. Close the lid gently, and centrifuge for 15 seconds at greater than or equal to 8000×g (greater than or equal to 10,000 rpm) to wash the spin column membrane. Discard the flow through.
12. Add 500 μl RPE Buffer to the RNeasy MinElute spin column. Close the lid gently, and centrifuge for 2 min at greater than or equal to 8000×g (greater than or equal to 10,000 rpm) to wash the spin column membrane. Discard the collection tube with the flow through.
13. Place the RNeasy MinElute spin column in a new 2 ml collection tube (supplied). Open the lid of the spin column, and centrifuge at full speed for 5 min. Discard the collection tube with the flow through. 14. Place the RNeasy MinElute spin column in a new 1.5 ml collection tube. Add 14-30 μl (we use 30 μl) RNase-free water directly to the spin column membrane. Close the lid gently, and centrifuge for 1 min at full speed to elute the RNA.

### Example 14. Isolation of Nuclei with NBT/BCIP Using Micropurification

Nuclei from human breast cancer FFPE tissue was successfully isolated via micropurification using a histone primary antibody and a secondary antibody labeled with alkaline phosphatase. The chromogen was NBT/BCIP. The standard IHC staining procedure described herein was used. For micropurification, a 1 mg/mL proteinase K and 1% SDS solution was contacted with the sample for about 10 minutes at 56° C. Corresponding images are shown in FIGS. 19A-19D.

### REFERENCES

[1] Brower V: NCI-MATCH pairs tumor mutations with matching drugs. Nature biotechnology 2015, 33:790-1.
[2] Cancer Genome Atlas N: Comprehensive molecular portraits of human breast tumours. Nature 2012, 490:61-70.
[3] Cancer Genome Atlas Research N: Comprehensive genomic characterization of squamous cell lung cancers. Nature 2012, 489:519-25.
[4] Cecchi F, Lih C J, Lee Y H, Walsh W, Rabe D C, Williams P M, Bottaro D P: Expression array analysis of the hepatocyte growth factor invasive program. Clinical & experimental metastasis 2015, 32:659-76.
[5] Frampton G M, Fichtenholtz A, Otto G A, Wang K, Downing S R, He J, Schnall-Levin M, White J, Sanford E M, An P, Sun J, Juhn F, Brennan K, Iwanik K, Maillet A, Buell J, White E, Zhao M, Balasubramanian S, Terzic S, Richards T, Banning V, Garcia L, Mahoney K, Zwirko Z, Donahue A, Beltran H, Mosquera J M, Rubin M A, Dogan S, Hedvat C V, Berger M F, Pusztai L, Lechner M, Boshoff C, Jarosz M, Vietz C, Parker A, Miller V A, Ross J S, Curran J, Cronin M T, Stephens P J, Lipson D, Yelensky R: Development and validation of a clinical cancer genomic profiling test based on massively parallel DNA sequencing. Nature biotechnology 2013, 31:1023-31.
[6] Fumagalli D, Gavin P G, Taniyama Y, Kim S I, Choi H J, Paik S, Pogue-Geile K L: A rapid, sensitive, reproducible and cost-effective method for mutation profiling of colon cancer and metastatic lymph nodes. BMC cancer 2010, 10:101.
[7] Garcia-Murillas I, Lambros M, Turner N C: Determination of HER2 amplification status on tumour DNA by digital PCR. PloS one 2013, 8:e83409.
[8] Grosserueschkamp F, Bracht T, Diehl H C, Kuepper C, Ahrens M, Kallenbach-Thieltges A, Mosig A, Eisenacher M, Marcus K, Behrens T, Bruning T, Theegarten D, Sitek B, Gerwert K: Spatial and molecular resolution of diffuse malignant mesothelioma heterogeneity by integrating label-free FTIR imaging, laser capture microdissection and proteomics. Scientific reports 2017, 7:44829.
[9] Kummar S, Williams P M, Lih C J, Polley E C, Chen A P, Rubinstein L V, Zhao Y, Simon R M, Conley B A, Doroshow J H: Application of molecular profiling in clinical trials for advanced metastatic cancers. J Natl Cancer Inst 2015, 107.
[10] Lih C J, Sims D J, Harrington R D, Polley E C, Zhao Y, Mehaffey M G, Forbes T D, Das B, Walsh W D, Datta V, Harper K N, Bouk C H, Rubinstein L V, Simon R M, Conley B A, Chen A P, Kummar S, Doroshow J H, Williams P M: Analytical Validation and Application of a Targeted Next-Generation Sequencing Mutation-Detection Assay for Use in Treatment Assignment in the NCI-MPACT Trial. J Mol Diagn 2015.
[11] Linton K, Howarth C, Wappett M, Newton G, Lachel C, Iqbal J, Pepper S, Byers R, Chan W J, Radford J: Microarray gene expression analysis of fixed archival tissue permits molecular classification and identification of potential therapeutic targets in diffuse large B-cell lymphoma. J Mol Diagn 2012, 14:223-32.
[12] Nielsen T, Wallden B, Schaper C, Ferree S, Liu S, Gao D, Barry G, Dowidar N, Maysuria M, Storhoff J: Analytical validation of the PAM50-based Prosigna Breast Cancer Prognostic Gene Signature Assay and nCounter Analysis System using formalin-fixed paraffin-embedded breast tumor specimens. BMC cancer 2014, 14:177.
[13] Scott D W, Wright G W, Williams P M, Lih C J, Walsh W, Jaffe E S, Rosenwald A, Campo E, Chan W C, Connors J M, Smeland E B, Mottok A, Braziel R M, Ott G, Delabie J, Tubbs R R, Cook J R, Weisenburger D D, Greiner T C, Glinsmann-Gibson B J, Fu K, Staudt L M, Gascoyne R D, Rimsza L M: Determining cellof-origin subtypes of diffuse large B-cell lymphoma using gene expression in formalin-fixed paraffin embedded tissue. Blood 2014, 123:1214-7.
[14] Sparano J A, Gray R J, Makower D F, Pritchard K I, Albain K S, Hayes D F, Geyer C E, Jr., Dees E C, Perez E A, Olson J A, Jr., Zujewski J, Lively T, Badve S S, Saphner T J, Wagner L I, Whelan T J, Ellis M J, Paik S, Wood W C, Ravdin P, Keane M M, Gomez Moreno H L, Reddy P S, Goggins T F, Mayer I A, Brufsky A M, Toppmeyer D L, Kaklamani V G, Atkins J N, Berenberg J L, Sledge G W: Prospective Validation of a 21-Gene Expression Assay in Breast Cancer. The New England journal of medicine 2015, 373:2005-14.
[15] Yan W, Shih J H, Rodriguez-Canales J, Tangrea M A, Ylaya K, Hipp J, Player A, Hu N, Goldstein A M, Taylor P R, Emmert-Buck M R, Erickson H S: Identification of unique expression signatures and therapeutic targets in esophageal squamous cell carcinoma. BMC Res Notes 2012, 5:73.
[16] Zhuang Z, Bertheau P, Emmert-Buck M R, Liotta L A, Gnarra J, Linehan W M, Lubensky I A: A microdissection technique for archival DNA analysis of specific cell populations in lesions <1 mm in size. The American journal of pathology 1995, 146:620-5.
[17] Bonner R F, Emmert-Buck M, Cole K, Pohida T, Chuaqui R, Goldstein S, Liotta L A: Laser capture microdissection: molecular analysis of tissue. Science 1997, 278:1481,3.
[18] Eberle F C, Hanson J C, Killian J K, Wei L, Ylaya K, Hewitt S M, Jaffe E S, Emmert-Buck M R, Rodriguez-Canales J: Immunoguided laser assisted microdissection techniques for DNA methylation analysis of archival tissue specimens. J Mol Diagn 2010, 12:394-401.
[19] Emmert-Buck M R, Bonner R F, Smith P D, Chuaqui R F, Zhuang Z, Goldstein S R, Weiss R A, Liotta L A: Laser capture microdissection. Science 1996, 274:998-1001.
[20] Emmert-Buck M R, Roth M J, Zhuang Z, Campo E, Rozhin J, Sloane B F, Liotta L A, Stetler-Stevenson W G: Increased gelatinase A (MMP-2) and cathepsin B activity in invasive tumor regions of human colon cancer samples. The American journal of pathology 1994, 145:1285-90.
[21] Fend F, Emmert-Buck M R, Chuaqui R, Cole K, Lee J, Liotta L A, Raffeld M: Immuno-LCM: laser capture microdissection of immunostained frozen sections for mRNA analysis. The American journal of pathology 1999, 154:61-6.
[22] Hipp J, Cheng J, Hanson J C, Yan W, Taylor P, Hu N, Rodriguez-Canales J, Tangrea M A, Emmert-Buck M R, Balis U: SIVQ-aided laser capture microdissection: A tool for high-throughput expression profiling. J Pathol Inform 2011, 2:19.
[23] Simone N L, Remaley A T, Charboneau L, Petricoin E F, 3rd, Glickman J W, Emmert-Buck M R, Fleisher T A, Liotta L A: Sensitive immunoassay of tissue cell proteins procured by laser capture microdissection. The American journal of pathology 2000, 156:445-52.
[24] Tangrea M A, Chuaqui R F, Gillespie J W, Ahram M, Gannot G, Wallis B S, Best C J, Linehan W M, Liotta L A, Pohida T J, Bonner R F, Emmert-Buck M R: Expression microdissection: operator independent retrieval of cells for molecular profiling. Diagn Mol Pathol 2004, 13:207-12.
[25] Tangrea M A, Hanson J C, Bonner R F, Pohida T J, Rodriguez-Canales J, Emmert-Buck M R: Immunoguided microdissection techniques. Methods Mol Biol 2011, 755:57-66.
[26] Fearon E R, Hamilton S R, Vogelstein B: Clonal analysis of human colorectal tumors. Science 1987, 238:193-7.
[27] Radford D M, Fair K, Thompson A M, Ritter J H, Holt M, Steinbrueck T, Wallace M, Wells S A, Jr., Donis-Keller H R: Allelic loss on a chromosome 17 in ductal carcinoma in situ of the breast. Cancer research 1993, 53:2947-9.
[28] Shibata D, Hawes D, Li Z H, Hernandez A M, Spruck C H, Nichols P W: Specific genetic analysis of microscopic tissue after selective ultraviolet radiation fractionation and the polymerase chain reaction. The American journal of pathology 1992, 141:539-43.
[29] Kovach J S, McGovern R M, Cassady J D, Swanson S K, Wold L E, Vogelstein B, Sommer S S: Direct sequencing from touch preparations of human carcinomas: analysis of p53 mutations in breast carcinomas. J Natl Cancer Inst 1991, 83:1004-9.
[30] Noguchi S, Motomura K, Inaji H, Imaoka S, Koyama H: Clonal analysis of predominantly intraductal carcinoma and precancerous lesions of the breast by means of polymerase chain reaction. Cancer research 1994, 54:1849-53.
[31] Park T W, Felix J C, Wright T C, Jr.: X chromosome inactivation and microsatellite instability in early and advanced bilateral ovarian carcinomas. Cancer research 1995, 55:4793-6.
[32] Chuaqui R F, Sanz-Ortega J, Vocke C, Linehan W M, Sanz-Esponera J, Zhuang Z, Emmert-Buck M R, Merino M J: Loss of heterozygosity on the short arm of chromosome 8 in male breast carcinomas. Cancer research 1995, 55:4995-8.
[33] Best C J, Gillespie J W, Yi Y, Chandramouli G V, Perlmutter M A, Gathright Y, Erickson H S, Georgevich L, Tangrea M A, Duray P H, Gonzalez S, Velasco A, Linehan W M, Matusik R J, Price D K, Figg W D, Emmert-Buck M R, Chuaqui R F: Molecular alterations in primary prostate cancer after androgen ablation therapy. Clin Cancer Res 2005, 11:6823-34.
[34] Chowdhuri S R, Xi L, Pham T H, Hanson J, Rodriguez-Canales J, Berman A, Rajan A, Giaccone G, Emmert-Buck M, Raffeld M, Filie A C: EGFR and KRAS mutation analysis in cytologic samples of lung adenocarcinoma enabled by laser capture microdissection. Mod Pathol 2011, 25:548-55.
[35] Chuaqui R F, Bonner R F, Best C J, Gillespie J W, Flaig M J, Hewitt S M, Phillips J L, Krizman D B, Tangrea M A, Ahram M, Linehan W M, Knezevic V, Emmert-Buck M R: Post-analysis follow-up and validation of microarray experiments. Nat Genet 2002, 32 Suppl: 509-14.
[36] Johann D J, Rodriguez-Canales J, Mukherjee S, Prieto D A, Hanson J C, Emmert-Buck M, Blonder J: Approaching solid tumor heterogeneity on a cellular basis by tissue proteomics using laser capture microdissection and biological mass spectrometry. J Proteome Res 2009, 8:2310-8.
[37] Jumper N, Hodgkinson T, Paus R, Bayat A: Site-specific gene expression profiling as a novel strategy for unravelling keloid disease pathobiology. PloS one 2017, 12:e0172955.
[38] Cubi R, Vembar S S, Biton A, Franetich J F, Bordessoulles M, Sossau D, Zanghi G, Bosson-Vanga H, Benard M, Moreno A, Dereuddre-Bosquet N, Le Grand R, Scherf A: Laser capture microdissection enables transcriptomic analysis of dividing and quiescent liver stages of Plasmodium relapsing species. 2017.
[39] Ebrahimi A, Jung M H, Dreyfuss J M, Pan H, Sgroi D, Bonner-Weir S, Weir G C: Evidence of stress in beta cells obtained with laser capture microdissection from pancreases of brain dead donors. Islets 2017, 9:19-29.
[40] Serova T A, Tikhonovich I A, Tsyganov V E: Analysis of nodule senescence in pea (Pisum sativum L.) using laser microdissection, real-time PCR, and ACC immunolocalization. Journal of plant physiology 2017, 212:29-44.
[41] Adebayo Michael A O, Ahsan N, Zabala V, Francois-Vaughan H, Post S, Brilliant K E, Salomon A R, Sanders J A, Gruppuso P A: Proteomic analysis of laser capture microdissected focal lesions in a rat model of progenitor marker-positive hepatocellular carcinoma. Oncotarget 2017.
[42] Costa S, Correia-de-Sa P, Porto M J, Caine L: The Use of Laser Microdissection in Forensic Sexual Assault Casework: Pros and Cons Compared to Standard Methods. Journal of forensic sciences 2017.
[43] Ning X, Yin Z, Li Z, Xu J, Wang L, Shen W, Lu Y, Cai G, Zhang X, Chen X: Comparative proteomic analysis of urine and laser microdissected glomeruli in IgA nephropathy. Clinical and experimental pharmacology & physiology 2017.
[44] Hobeika L, Barati M T, Caster D J, McLeish K R, Merchant M L: Characterization of glomerular extracellular matrix by proteomic analysis of laser-captured microdissected glomeruli. Kidney international 2017, 91:501-11.
[45] Mollee P, Boros S, Loo D, Ruelcke J E, Lakis V A, Cao K L, Renaut P, Hill M M: Implementation and evaluation of amyloidosis subtyping by laser-capture microdissection and tandem mass spectrometry. Clinical proteomics 2016, 13:30.
[46] Zhang L, Lanzoni G, Battarra M, Inverardi L, Zhang Q: Proteomic profiling of human islets collected from frozen pancreata using laser capture microdissection. Journal of proteomics 2017, 150:149-59.
[47] Gillespie J W, Gannot G, Tangrea M A, Ahram M, Best C J, Bichsel V E, Petricoin E F, Emmert-Buck M R, Chuaqui R F: Molecular profiling of cancer. Toxicol Pathol 2004, 32 Suppl 1:67-71.
[48] Erickson H S, Gillespie J W, Emmert-Buck M R: Tissue microdissection. Methods Mol Biol 2008, 424:433-48.
[49] Bova G S, Eltoum I A, Kiernan J A, Siegal G P, Frost A R, Best C J, Gillespie J W, Su G H, Emmert-Buck M R: Optimal molecular profiling of tissue and tissue components: defining the best processing and microdissection methods for biomedical applications. Mol Biotechnol 2005, 29:119-52.
[50] Rosenberg A Z, Armani M D, Fetsch P A, Xi L, Pham T T, Raffeld M, Chen Y, O'Flaherty N, Stussman R, Blackler A R, Du Q, Hanson J C, Roth M J, Filie A C, Roh M H, Emmert-Buck M R, Hipp J D, Tangrea M A: High-Throughput Microdissection for Next-Generation Sequencing. PloS one 2016, 11:e0151775.
[51] Quiros P M, Goyal A, Jha P, Auwerx J: Analysis of mtDNA/nDNA Ratio in Mice. Current protocols in mouse biology 2017, 7:47-54.
[52] Evdokimovskii E V, Gubina N E, Ushakova T E, Gaziev A I: [Changes of mitochondrial DNA/nuclear DNA ratio in the blood serum following X-ray irradiation of mice at various doses]. Radiatsionnaia biologiia, radioecologiia 2012, 52:565-71.
[53] Kampira E, Dzobo K, Kumwenda J, van Oosterhout J J, Parker M I, Dandara C: Peripheral blood mitochondrial DNA/nuclear DNA (mtDNA/nDNA) ratio as a marker of mitochondrial toxicities of stavudine containing antiretroviral therapy in HIV-infected Malawian patients. Omics: a journal of integrative biology 2014, 18:438-45.
[54] Allio R, Donega S, Galtier N, Nabholz B: Large Variation in the Ratio of Mitochondria) to Nuclear Mutation Rate across Animals: Implications for Genetic Diversity and the Use of Mitochondria) DNA as a Molecular Marker. Molecular biology and evolution 2017, 34:2762-72.

1. A method of collecting cells comprising:
contacting a biological sample with a specific probe to form a bound complex comprising cells labeled with said specific probe, wherein said specific probe comprises a reactive moiety that deposits a protective barrier onto the labeled cells or subcellular structures, or carries a chemical, physical, or molecular protective barrier that covers the labeled cells or subcellular structures;
applying a micropurification solution to the biological sample, wherein said micropurification solution degrades, digests, or otherwise differentially processes cells or structures not protected by the barrier.

2. The method of claim 1, wherein the specific probe comprises a reactive moiety, further comprising:
contacting said bound complex comprising the reactive moiety with a substrate that reacts with said reactive moiety to deposit a chemical material onto the surface of said cells labeled with said specific probe, wherein said chemical material forms a barrier covering the labeled cells or subcellular structures.

3. The method of claim 1, wherein a plurality of specific probes is used, and wherein each probe binds to a different target.

4. The method of claim 1, wherein said reactive moiety comprises an enzyme and wherein said substrate is a substrate for said enzyme.

5. The method of claim 1 wherein said specific probe comprises an antibody.

6. The method of claim 5, wherein said probe comprises a primary antibody and a secondary antibody, wherein said primary antibody specifically binds specific cells or cellular structures in said tissue, and wherein said secondary antibody specifically binds said primary antibody and comprises an enzyme capable of reacting with said substrate to form said barrier.

7. The method of claim 5, wherein the antibody binds to a tumor marker, a cancer marker, a nuclear marker, an extracellular marker, an intracellular marker, or an immunological marker.

8. The method of claim 1, further comprising collecting cells covered by the barrier.

9. The method of claim 8, wherein the cells collected are selected from the group consisting of neoplastic cells, cancer cells, tumor cells, immune cells, normal cells, subcellular compartments, and structural components of a tissue.

10. The method of claim 1, further comprising collecting cells that are not covered by the barrier.

11. The method of claim 1, wherein said probe comprises a nucleotide sequence.

12. The method of claim 11 wherein said reactive moiety is an enzyme that reacts with a chromophore to produce an insoluble chemical material.

13. The method of claim 11, wherein said probe is complementary to a DNA sequence comprising a mutation.

14. The method of claim 1, wherein said probe comprises an affimer or an aptamer.

15. The method of claim 14, wherein said reactive moiety is an enzyme that reacts with a chromophore to produce an insoluble chemical material.

16. The method of claim 1, wherein said substrate is a chromogen that is processed by a peroxidase enzyme.

17. (canceled)

18. The method of claim 1, wherein said substrate is a chromogen that is processed by a phosphatase enzyme.

19. (canceled)

20. The method of claim 8, further comprising isolating DNA from the collected cells and determining a DNA allelotype.

21. The method of claim 1, wherein the cells are affixed to a surface or embedded in paraffin.

22. The method of claim 8, further comprising analyzing the collected cells using a method comprising one or more of next generation sequencing (NGS), expression profiling, proteomic analysis, lipid analysis, metabolic analysis, and/or immunological activity analysis.

23. (canceled)
